(19) United States
(12) Patent Application Publication
(10) Pub. No.: US 2024/0353532 A1
(43) Pub. Date: Oct. 24, 2024

(54) SYSTEMS AND METHODS FOR OPTICAL TRACKING

(71) Applicant: Meta Platforms Technologies, LLC, Menlo Park, CA (US)

(72) Inventors: John Ho, Sunnyvale, CA (US); Jiang Zhu, Cupertino, CA (US); Boon Shiu, Palo Alto, CA (US); Paurakh Rajbhandary, San Jose, CA (US); Yuge Huang, Painted Post, NY (US); Lu Lu, Kirkland, WA (US); Junren Wang, Redmond, WA (US); Mengfei Wang, Woodinville, WA (US); Xiayu Feng, Kirkland, WA (US); Zhexin Zhao, Redmond, WA (US); Steven Alexander-Boyd Hickman, Seattle, WA (US); Tingling Rao, Bellevue, WA (US); Kimberly Kay Childress, Duvall, WA (US); Amir Shariffar, Redmond, WA (US); Sadegh Aghaei, Worcester, MA (US); Zhaoyu Nie, Redmond, WA (US); Prathmesh Deshmukh, Redmond, WA (US); Zhaocheng Liu, Redmond, WA (US); Raymond Smith, II, Spartanburg, SC (US); Andrew John Ouderkirk, Kirkland, WA (US); Sawyer Miller, Bellevue, WA (US); Hsien-Hui Cheng, Woodinville, WA (US); Xuan Wang, Kirkland, WA (US); Ali Altaqui, Raleigh, NC (US); Zhuoliang Ni, Kenmore, WA (US)

(21) Appl. No.: 18/642,484

(22) Filed: Apr. 22, 2024

Related U.S. Application Data
(60) Provisional application No. 63/497,400, filed on Apr. 20, 2023.
(60) Provisional application No. 63/513,494, filed on Jul. 13, 2023.
(60) Provisional application No. 63/595,919, filed on Nov. 3, 2023.
(60) Provisional application No. 63/598,966, filed on Nov. 15, 2023.
(60) Provisional application No. 63/598,967, filed on Nov. 15, 2023.

(51) Int. Cl. G01S 7/41; A61B 5/024; G02B 27/01; G03H 1/02

(52) U.S. Cl. CPC G01S 7/412; A61B 5/024; G01S 7/415; G02B 27/0172; G03H 1/0248; G02B 2027/0174; G02B 2027/0178; G03H 2001/026; G03H 2250/38

(57) ABSTRACT

A computer-implemented method may include (1) causing a radar component to emit one or more radar signals and (2) causing the radar component to analyze one or more return signals. Also disclosed is a method for forming a 3D liquid crystal polarization hologram optical element and a method for characterizing diffractive waveguides includes directing light onto a structure and measuring the diffracted light to capture at least one image of the structure. Lastly, disclosed is a method of pattering organic solid crystals and a method directing a beam of input light to a surface of an optical material to determine crystallographic and optical parameters of the optical material. Various other methods, systems, and computer-readable media are also disclosed.

### CROSS-REFERENCE TO RELATED APPLICATIONS

This application claims priority to U.S. Application 63/497,400, filed 20 Apr. 2023, U.S. Application No. 63/513,494, filed 7 Jul. 2023, U.S. Application No. 63/595,919, filed 11 Nov. 2023, U.S. Application No. 63/598,966, filed 15 Nov. 2023, and U.S. Application No. 63/598,967, filed 15 Nov. 2023, the disclosures of each of which are incorporated, in their entirety, by this reference.

### BRIEF DESCRIPTION OF THE DRAWINGS

The accompanying drawings illustrate a number of exemplary embodiments and are a part of the specification. Together with the following description, these drawings demonstrate and explain various principles of the present disclosure.

FIG. 1 is a flow diagram of an exemplary method for calibrating and training radar-based tracking systems.

FIG. 2 is a block diagram of an exemplary system for calibrating and training radar-based tracking systems.

FIG. 3 is an illustration of an exemplary reflector altering the field of view of a radar component (e.g., radar sensor).

FIG. 4 is a block diagram of an exemplary radar signal being emitted by a transmitter of a radar component, reflected off an object, and received as a return signal by a receiver of the radar component.

FIG. 5 is a block diagram of a process for training a machine learning model.

FIG. 6 is a schematic view of visible and/or near-IR two-photon polymerization processes according to some embodiments.

FIG. 7 is a perspective view showing volumetric and surface photopolymerization and alignment techniques according to certain embodiments.

### Appendix A

FIG. 8 is a diagram of an embossing apparatus according to some embodiments.

FIG. 9 is a diagram of an embossing apparatus heating a thermal device according to some embodiments.

FIG. 10 is a diagram of an embossing apparatus locally heating an OCS surface according to some embodiments.

FIG. 11 is a diagram of an embossing apparatus removing portions of an OCS surface, according to some embodiments.

FIG. 12 is a diagram of an embossing apparatus controlling the depth of features within an OCS surface, according to some embodiments.

FIG. 13 is a diagram of an embossing apparatus maintain a constant distance between a thermal device and an OCS surface, according to some embodiments.

FIG. 14 is a diagram of an embossing apparatus withdrawing a thermal device from an OCS surface, according to some embodiments.

FIG. 15 is an isometric view depicting characteristic optical and crystallographic properties of an optically anisotropic thin film according to some embodiments.

FIG. 16 is a schematic view of a metrology apparatus for evaluating optical and crystallographic properties of an optically anisotropic material according to some embodiments.

FIG. 17 is a schematic view of a metrology apparatus for evaluating optical and crystallographic properties of an optically anisotropic material according to certain embodiments.

FIG. 18 illustrates a technique for characterizing the optical and crystallographic properties of an optically anisotropic material according to further embodiments.

FIG. 19 is a flowchart detailing aspects of a process for evaluating optical and crystallographic parameters of an anisotropic material according to some embodiments.

FIG. 20 is an illustration of exemplary augmented-reality glasses that may be used in connection with embodiments of this disclosure.

FIG. 21 is an illustration of an exemplary virtual-reality headset that may be used in connection with embodiments of this disclosure.

Throughout the drawings, identical reference characters and descriptions indicate similar, but not necessarily identical, elements. While the exemplary embodiments described herein are susceptible to various modifications and alternative forms, specific embodiments have been shown by way of example in the drawings and will be described in detail herein. However, the exemplary embodiments described herein are not intended to be limited to the particular forms disclosed. Rather, the present disclosure covers all modifications, equivalents, and alternatives falling within the scope of the appended claims.

### DETAILED DESCRIPTION OF EXEMPLARY EMBODIMENTS

The embodiments herein provide systems that use radar sensors (e.g., close-range radar sensors) to perform facial tracking (and/or tracking of other body parts). Radar sensors may penetrate hair, masks, and other objects, perform well in ambient lighting, and have low power consumption (e.g., close-range sensors may operate at approximately 1-5 mW). As such, the radar sensing embodiments described herein may address pain points previously experienced in camera-based face tracking (e.g., due to hair occlusions, poor upper and lower face visibility, variable lighting conditions, and power consumption needs).

The disclosed radar tracking systems may implement different methods of analyzing radar data to identify features on a user's face and/or track the movement of those features, as will be discussed later. Other potential embodiments include using radar data to detect a user's heartbeat (e.g., at the user's temples) or to detect respiration rates (e.g., based on chest movements). Still further, in some cases, metallic reflectors may be specifically designed and placed on an AR device to broaden (or focus) the field of view of each radar sensor. This may allow the sensors to provide more precise and accurate sensing data which, in turn, may allow the radar facial tracking system to track smaller movements and be more precise in its identified movements.

The barriers to using radar tracking systems for feature identification and/or tracking (e.g., face tracking) are many. Radar sensing has low spatial resolution (e.g., set by diffraction limit and bandwidth). In some cases, data acquired by radar sensors may not be human-interpretable and may sense fundamentally different physical parameters than cameras (e.g., depth, displacement, velocity, angle, etc., as compared to a camera's measurement of optical contrast). Traditional radar processing methods are not suited for face tracking because they focus on object detection and localization (depth and lateral resolution)—as opposed to the relative pose estimation (displacement sensitivity) enabled in the radar tracking framework described herein. This disclosure responds to these barriers with a radar tracking system that translates radar data into facial landmarks using depth, displacement, and velocity information, as will be described in greater detail below. In some examples, the radar tracking system may train a machine learning (ML) model using camera data and may subsequently learn to customize facial (and/or body) tracking for each user's face.

This disclosure is generally directed to a facial tracking system that uses radar instead of or together with camera-based facial tracking. In current facial tracking systems, many cameras may be required to perform reliable face tracking. Each of these cameras typically operates at more than 10 mW. In the artificial reality (AR) context, these cameras require significant space on an AR device. Still further, cameras cannot track facial movements through facial hair, bangs, masks, or other impediments and they do not work well under low-light conditions.

As applied to the display of three-dimensional (3D) images, and in contrast with comparative display systems, holography can record and reconstruct the amplitude and phase of an optical wavefront and may be configured to display real-time 3D images in free space with technically relevant visual depth cues, including shadowing, texture, motion parallax, occlusion, and ocular accommodation.

Holographic display technology may be configured to reconstruct a 3D replica of an original object with high fidelity. As liquid crystal materials can modulate the polarization state of light, they may be incorporated into a holographic display, such as, for example, a liquid crystal spatial light modulator or a liquid crystal lens, to modulate the phase and amplitude of optical information. In contrast to comparative geometric lenses, for instance, which may have a thickness on the order of millimeters, LC holographic lenses may have a thickness on the order of micrometers.

Notwithstanding recent developments, it would be advantageous to develop improved methods for the 3D patterning of liquid crystal media for hologram recording and image reconstruction. Disclosed are methods for fabricating a holographic display system. Particular embodiments relate to methods for 3D patterning of suitable liquid crystal display media.

In accordance with various embodiments, an LC holographic medium may have an arbitrary director profile, including a birefringent refractive index profile with an arbitrary optical axis, within a 3D volume with a patterning resolution of approximately 30-250 nm for light modulation across the visible and near IR spectra.

As will be appreciated, LC molecules may respond to incident light and, depending on the choice of the LC material, align from a random configuration to be oriented either parallel or perpendicular to the light's local linear polarization. That is, the LC medium may be configured to record the linear polarization of an incident light beam where the polarization of the incident beam at a focal point within the LC medium may be spatially and temporally controlled.

Example LC materials include photo-responsive reactive mesogens, including transmissive polarization volume hologram (tPVH) materials, and mixtures of photo-responsive reactive mesogens with dopants such as chiral compounds, dichroic photo-initiators and/or dichroic dyes.

In a point-by-point (0D) patterning method, the LC molecular orientation within a 3D volume is written pixel-by-pixel. Point-by-point patterning may include a two-photon polymerization (TPP) configuration, for example, where light at the focal point has a predetermined linear polarization to induce LC birefringence. Two-photon polymerization, also known as direct laser writing, is a non-linear optical process based on the simultaneous absorption of two photons by a photosensitive material.

In certain implementations, TPP optics may be fixed, and a sample may be translated relative to the processing optical beam. Two-photon polymerization may be configured to fabricate 3D structures with dimensions ranging from hundreds of nanometers to a few hundred micrometers. Point-by-point patterning may be applied to mask fabrication, for example.

Slit-by-slit (1D) patterning may include polarization variation along a single dimension, e.g., p(x), and may use a cylindrical lens setup to form focal slits for faster patterning speeds than achievable with point-by-point patterning. In certain instantiations, at the focal slit, light may exhibit varying linear polarization along the slit direction. With a slit-by-slit approach, the projection optics and choice of the light source may impact the sharpness of the “slit” intensity profile and the quality of the linear polarization at the focal slit.

Layer-by-layer (2D) patterning may include polarization variation along two dimensions, e.g., p(x,y). An example layer-by-layer process may include repetition of coating, patterning, and optional annealing steps. A layer-by-layer architecture may include plural stacked photo-responsive materials with different or equivalent patterning in each layer. Bulk photoalignment materials used in conjunction with layer-by-layer patterning may exhibit high photostability and may, for example, exhibit less than approximately 5% birefringence at 20× UV overexposure. According to further embodiments, a method may include stacking multiple LC gratings without intervening photoalignment layers where each LC grating may have different or equivalent in-plane periodicity.

Volumetric (3D) patterning may include polarization variation along three dimensions, e.g., p (x, y, z). Volumetric patterning may use a bulk layer of a photoalignment material with a single exposure on a relatively thin (less than approximately 5 micrometers) LC film. With volumetric patterning, an arbitrary linear polarization may be coded in a beam using vector beam shaping. For instance, a layer of photo-responsive material may be exposed to a beam having a programmed 3D varying linear polarization.

As will be appreciated, with the foregoing methods, throughout an act of polymerization and photoalignment, characteristics of the processing light (i.e., intensity, polarization, wavelength, focal point size and shape, etc.) may be fixed or variable. In certain instantiations, due to elastic forces and inter-molecular coupling, irradiated LC molecules may induce the alignment of unirradiated neighboring molecules, which may influence high-frequency director variation and limit patterning resolution.

In some embodiments, the disclosed methods may be used to fabricate LC films that are compatible with non-planar substrates. In further embodiments, the disclosed methods may be used to fabricate a compensation layer that may be used to correct geometrical lens aberrations. In various aspects, the disclosed methods may add a degree of freedom to the design of certain optical devices and systems, for instance, in a manner that decreases the number of layers in broadband and wide-view LC gratings and lenses having a multilayer structure. The disclosed methods may be used to fabricate an angular filter that mitigates “rainbow” visual artifacts, such as for AR displays. For example, such a filter may be configured to transmit light within a ±30° window from normal incidence and absorb or reflect light outside of that window.

Polymer and other organic materials may be incorporated into a variety of different optic and electro-optic device architectures, including passive and active optics and electroactive devices. Lightweight and conformable, one or more polymer/organic solid layers may be incorporated into wearable devices such as smart glasses and are attractive candidates for emerging technologies including virtual reality/augmented reality devices where a comfortable, adjustable form factor is desired.

Virtual reality (VR) and augmented reality (AR) eyewear devices or headsets, for instance, may enable users to experience events, such as interactions with people in a computer-generated simulation of a three-dimensional world or viewing data superimposed on a real-world view. By way of example, superimposing information onto a field of view may be achieved through an optical head-mounted display (OHMD) or by using embedded wireless glasses with a transparent heads-up display (HUD) or augmented reality (AR) overlay. VR/AR eyewear devices and headsets may be used for a variety of purposes. Governments may use such devices for military training, medical professionals may use such devices to simulate surgery, and engineers may use such devices as design visualization aids.

Organic solid crystal (OSC) materials with high refractive index and birefringence can be used for various optical components, including surface relief gratings, meta-surfaces, waveguides, beam splitting, photonic elements such as photonic integrated circuits, and polarization selective elements. For instance, an augmented reality display may include an OSC-based waveguide.

Organic solid crystals with high refractive index and birefringence have a unique value proposition for use in diffractive optical elements, such as a planar diffractive waveguide. An example waveguide includes a longitudinally extending high-index optical medium, which is transversely encased by low-index media or cladding. During use, a guided optical wave propagates in the waveguide through the high-index core along the longitudinal direction. In accordance with various embodiments, the high-index core of such a waveguide may be formed from an organic solid crystal (OSC). Such a construction may beneficially impact one or more of the display field of view, uniformity, efficiency, and cost of manufacture, etc.

Notwithstanding recent developments, it would be advantageous to provide a system and method for evaluating the crystallographic and optical properties of an OSC material, including principal refractive indices and, in the case of single crystal materials, the crystalline orientation with respect to a reference coordinate.

One or more source materials may be used to form an organic solid crystal, including an OSC substrate. Example organic materials include various classes of crystallizable organic semiconductors. Organic semiconductors may include small molecules, macromolecules, liquid crystals, organometallic compounds, oligomers, and polymers. Organic semiconductors may include p-type, n-type, or ambipolar polycyclic aromatic hydrocarbons, such as anthracene, phenanthrene, polycene, triazole, tolane, thiophene, pyrene, corannulene, fluorene, biphenyl, ter-phenyl, etc. Further example small molecules include fullerenes, such as carbon 60.

Example compounds may include cyclic, linear and/or branched structures, which may be saturated or unsaturated, and may additionally include heteroatoms and/or saturated or unsaturated heterocycles, such as furan, pyrrole, thiophene, pyridine, pyrimidine, piperidine, and the like. Heteroatoms (e.g., dopants) may include fluorine, chlorine, nitrogen, oxygen, sulfur, phosphorus, as well as various metals. Suitable feedstock for depositing solid organic semiconductor materials may include neat organic compositions, melts, solutions, or suspensions containing one or more of the organic materials disclosed herein.

Such organic materials may provide functionalities, including phase modulation, beam steering, wave-front shaping and correction, optical communication, optical computation, holography, and the like. Due to their optical and mechanical properties, organic solid crystals may enable high-performance devices, may be incorporated into passive or active optics, including AR/VR headsets, and may replace comparative material systems such as polymers, inorganic materials, and liquid crystals. In certain aspects, organic solid crystals may have optical properties that rival those of inorganic crystals while exhibiting the processability and electrical response of liquid crystals.

Structurally, the disclosed organic materials may be glassy, polycrystalline, or single crystal. Organic solid crystals, for instance, may include closely packed structures (e.g., organic molecules) that exhibit desirable optical properties such as a high and tunable refractive index, and high birefringence. Optically anisotropic organic solid materials may include a preferred packing of molecules, i.e., a preferred orientation or alignment of molecules. Example devices may include a birefringent organic solid crystal thin film or substrate having a high refractive index that may be further characterized by a smooth exterior surface.

High refractive index and highly birefringent organic semiconductor materials may be manufactured as a free-standing article or as a thin film deposited onto a substrate. An epitaxial or non-epitaxial growth process, for example, may be used to form an organic solid crystal (OSC) layer over a suitable substrate or within a mold. A seed layer for encouraging crystal nucleation and an anti-nucleation layer configured to locally inhibit nucleation may collectively promote the formation of a limited number of crystal nuclei within specified locations, which may in turn encourage the formation of larger organic solid crystals.

As used herein, the terms “epitaxy,” “epitaxial” and/or “epitaxial growth and/or deposition” refer to the nucleation and growth of an organic solid crystal on a deposition surface where the organic solid crystal layer being grown assumes the same crystalline habit as the material of the deposition surface. For example, in an epitaxial deposition process, chemical reactants may be controlled, and the system parameters may be set so that depositing atoms or molecules alight on the deposition surface and remain sufficiently mobile via surface diffusion to orient themselves according to the crystalline orientation of the atoms or molecules of the deposition surface. An epitaxial process may be homogeneous or heterogeneous.

Organic solid crystals may be characterized by six fundamental parameters, which are the refractive indices (nₓ, n_{y}, n_{z}) along first, second, and third principal axes and the optical orientation (aₓ, a_{y}, a_{z}) of each of the first, second, and third principal axes themselves. In some embodiments, the organic crystalline phase may be optically isotropic (n1=n2=n3) or birefringent, where n1≠n2≠n3, or n1≠n2=n3, or n1=n2≠n3, and may be characterized by a birefringence (Dn) between at least one pair of orientations of at least approximately 0.1, e.g., at least approximately 0.1, at least approximately 0.2, at least approximately 0.3, at least approximately 0.4, or at least approximately 0.5, including ranges between any of the foregoing values. In some embodiments, a birefringent organic crystalline phase may be characterized by a birefringence of less than approximately 0.1, e.g., less than approximately 0.1, less than approximately 0.05, less than approximately 0.02, less than approximately 0.01, less than approximately 0.005, less than approximately 0.002, or less than approximately 0.001, including ranges between any of the foregoing values.

An OSC substrate may have principal refractive indices (n1, n2, n3) where n1, n2, and n3 may independently vary from approximately 1.0 to approximately 4.0. According to further embodiments, an organic solid crystal may be characterized by a refractive index along at least one direction (i.e., along one direction, along a pair of orthogonal directions, or along 3 mutually orthogonal directions) of at least approximately 1.8, e.g., 1.8, 1.9, 2.0, 2.1, 2.2, 2.3, 2.4, 2.5, 2.6, or 2.7, including ranges between any of the foregoing values.

Organic solid crystal materials, including multilayer organic solid crystal thin films or substrates, may be optically transparent and exhibit low bulk haze. As used herein, a material or element that is “transparent” or “optically transparent” may, for a given thickness, have a transmissivity within the visible light spectrum of at least approximately 80%, e.g., approximately 80, 90, 95, 97, 98, 99, or 99.5%, including ranges between any of the foregoing values, and less than approximately 5% bulk haze, e.g., approximately 0.1, 0.2, 0.4, 1, 2, or 4% bulk haze, including ranges between any of the foregoing values. Transparent materials will typically exhibit very low optical absorption and minimal optical scattering.

As used herein, the terms “haze” and “clarity” may refer to an optical phenomenon associated with the transmission of light through a material, and may be attributed, for example, to the refraction of light within the material, e.g., due to secondary phases or porosity and/or the reflection of light from one or more surfaces of the material. As will be appreciated, haze may be associated with an amount of light that is subject to wide angle scattering (i.e., at an angle greater than 2.5° from normal) and a corresponding loss of transmissive contrast, whereas clarity may relate to an amount of light that is subject to narrow angle scattering (i.e., at an angle less than 2.5° from normal) and an attendant loss of optical sharpness or “see through quality.”

As used herein, in connection with a waveguide that includes a grating configured to couple light into or out of the waveguide, a grating is an optical element having a periodic structure that is configured to disperse or diffract light into plural component beams. The direction or diffraction angles of the diffracted light may depend on the wavelength of the light incident on the grating, the orientation of the incident light with respect to a grating surface, and the spacing between adjacent diffracting elements. In certain embodiments, grating architectures may be tunable along one, two, or three dimensions.

A grating may overlie a substrate through which an electromagnetic wave may propagate. According to various embodiments, the substrate may include or may be formed from an organic solid crystal material. The OSC material may be single crystal or polycrystalline and may include an amorphous organic phase. In some examples, the substrate may include a single phase OSC material. In some examples, the substrate may include a single organic solid crystal layer or an OSC multilayer. Each OSC layer may be characterized by three principal refractive indices, where n₁≠n₂≠n₃, n₁=n₂≠n₃, n₁≠n₂=n₃, n₁=n₃≠n₂. The refractive indices (n₁, n₂, n₃) may be aligned or askew with respect to the principal dimensions of the substrate.

An optical element may include a grating disposed over an OSC substrate. The grating may include a plurality of raised structures and may constitute a surface relief grating, for example. Gratings may be configured as binary or slanted gratings, for example, having a polar angle (q) and an azimuthal angle (j), where 0≤θ≤π and φ (0≤φ≤π). An OSC substrate may include an OSC material with either a fixed optical axis or a spatially varying optical axis.

An optical element may be formed by depositing a blanket layer of an organic solid crystal over a substrate or by providing an OSC substrate followed by photolithography and etching to define the raised structures. In alternate embodiments, individual raised structures may be printed or formed separately and then laminated to the substrate. Such structures may be sized and dimensioned to define a 1D or 2D periodic or non-periodic grating.

In some systems, the illumination path may include free space light (single and multi-wavelength) illumination. In particular embodiments, a high index prism may be located within the illumination path in contact with the OSC sample. The imaging path may include Fourier imaging or magnified or demagnified real space imaging. In accordance with various embodiments, the optimization pipeline may include optimization and retrieval based on polarization ray splitting of ordinary and extraordinary rays, including optimization and retrieval based on Brewster angle or optimization and retrieval based on critical angle.

In an example method, imaging optics directs light having a known polarization onto a sample at a known location of the sample. The imaging optics then measures the location of light exiting the sample. The location of transmitted or reflected light may be impacted by the refractive indices and orientation of the sample, which may include the bifurcation of a single input beam into different polarization states and the generation of a pair of output beams. The location of transmitted or reflected light may be determined for different angles (i.e., polar and/or azimuthal angles) of the incident light. For instance, as a function of the angle of incidence, a birefringent sample may be characterized by a pair of critical angles or Brewster angles corresponding to respective index directions. Using the input and measured characteristics of light interacting with the sample, an algorithm may determine the optical and crystalline properties of the sample.

This disclosure is generally directed to a facial tracking system that uses radar instead of or together with camera-based facial tracking. In current facial tracking systems, many cameras may be required to perform reliable face tracking. Each of these cameras typically operates at more than 10 mW. In the artificial reality (AR) context, these cameras require significant space on an AR device. Still further, cameras cannot track facial movements through facial hair, bangs, masks, or other impediments and they do not work well under low-light conditions.

Also, disclosed are methods for locally defining the orientation of LC molecules within a bulk volume. The various methods may be applied to align liquid crystals within a 3D structure in accordance with a target configuration. Approaches for 3D patterning of the LC medium may include pixel-by-pixel or layer-by-layer paradigms and may include (a) point-by-point patterning, (b) slit-by-slit patterning, (c) layer-by-layer patterning, and (d) volumetric patterning. Example methods thus contemplate the physical rendering of a prescribed computer-generated design, for example. Similarly disclosed are methods for polarization-based imaging (e.g., Mueller or Stokes based imaging) to allow for an entire area of a sample to be captured in single or multiple shots in a full-aperture measurement. Emerging technologies suggest using a combination of polarization-based imaging and computer simulations to characterize essential attributes of a waveguide. Example attributes may include spatial non-uniformities such as varying slant, chiral pitch, thickness, etc . . . . Further, the methods described may enable quick measurements over large areas, as opposed to comparative methods where two-dimensional raster scanning of point measurements may take much longer.

Also disclosed are systems and methods for changing the surface structure of organic solid crystal (OSC) materials. The inventive concept suggests using a source of patterned thermal energy (e.g., thermal device) to transfer thermal energy to the surface of the OSC material to excavate a portion of the OSC. The result of using this device includes the transfer of a pattern from the thermal device onto the surface of the OSC, while leaving the remaining OSC material in crystalline form. Similarly disclosed is a systematic method for evaluating the crystal indices and orientation of OSC materials. According to various embodiments, a metrology system includes a collimated illumination path with provisions for controlling the angle of incidence of input light onto an OSC sample, an imaging path to perform angle and intensity measurements of output light, and a digital optimization pipeline for determining crystal indices and orientation.

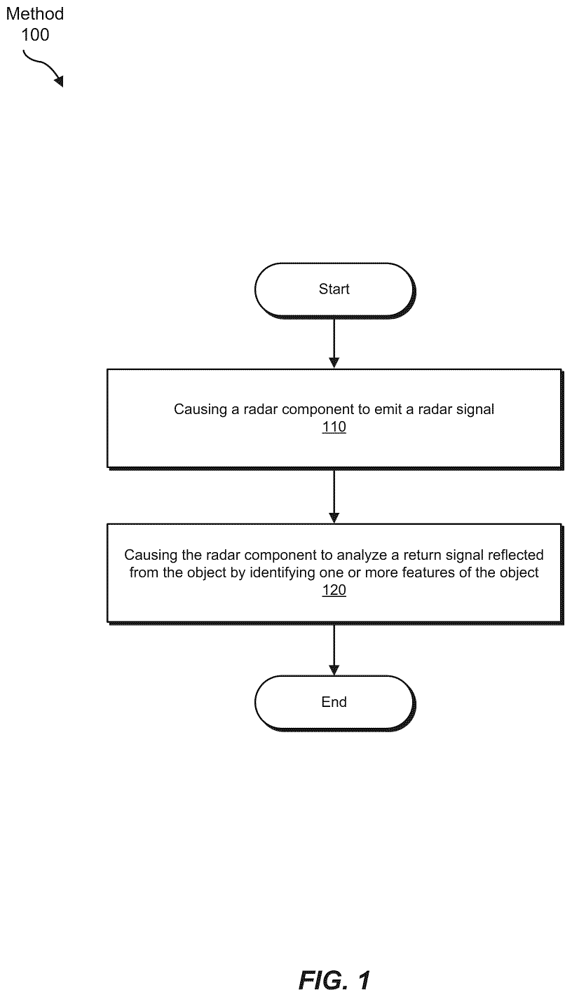

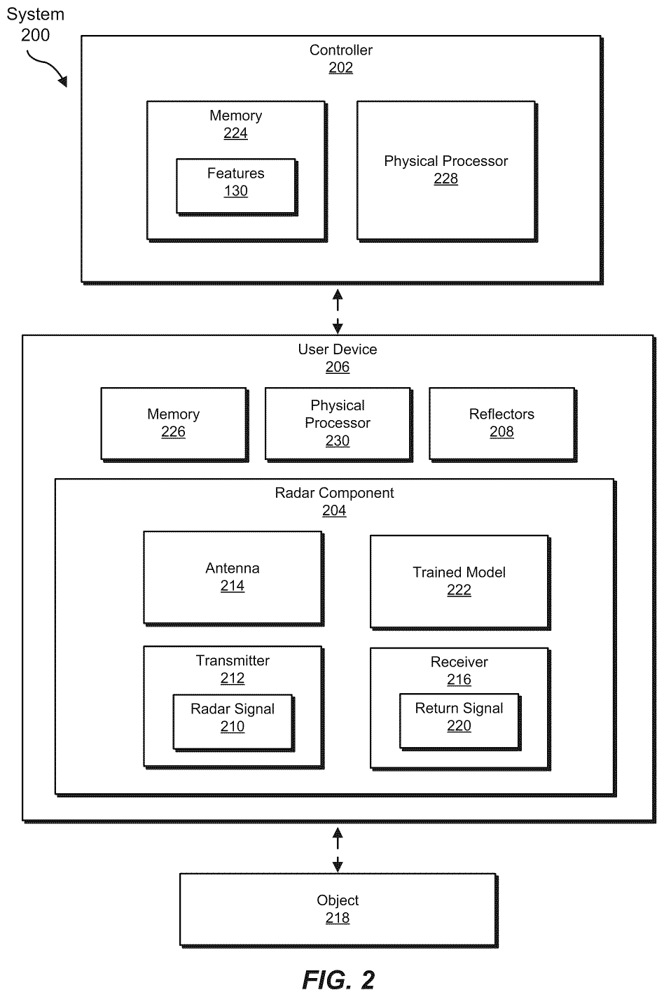

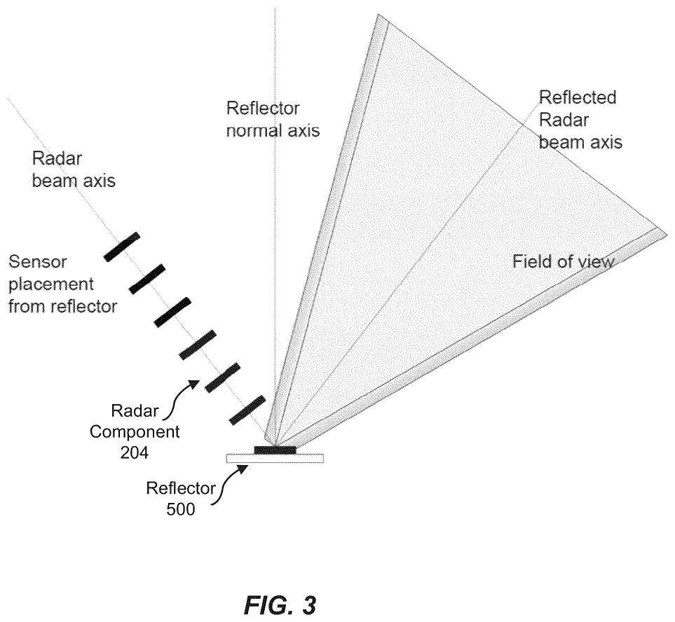

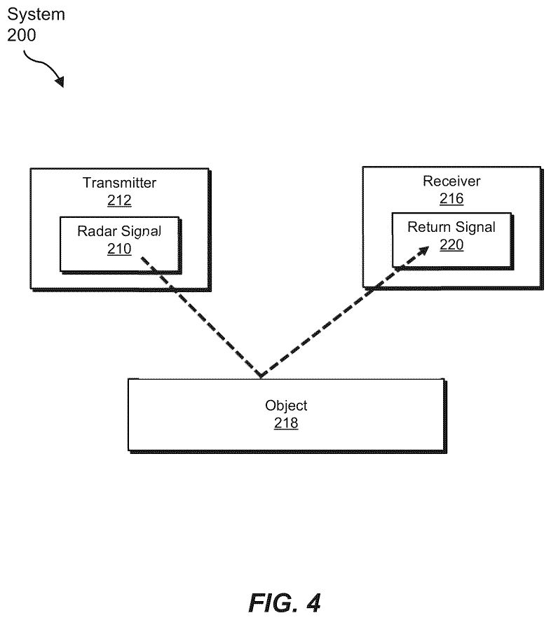

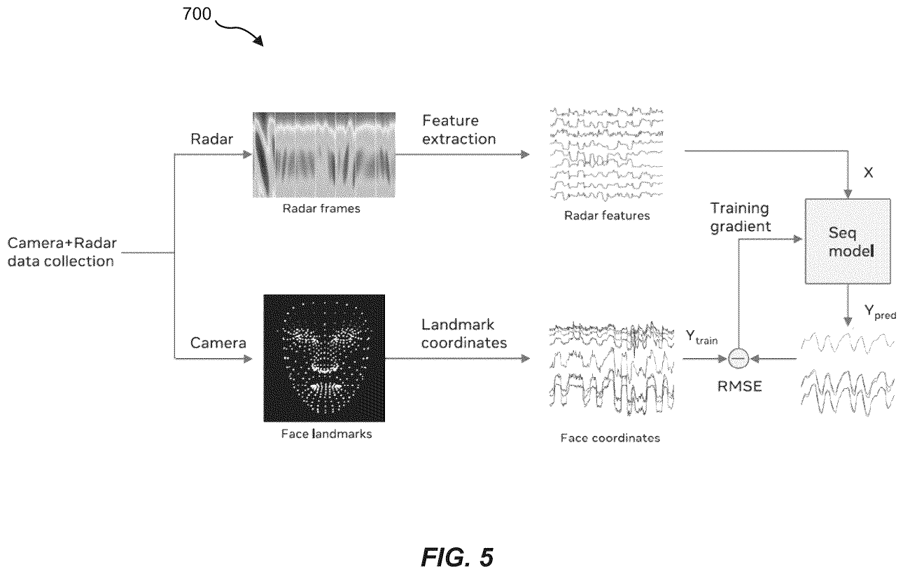

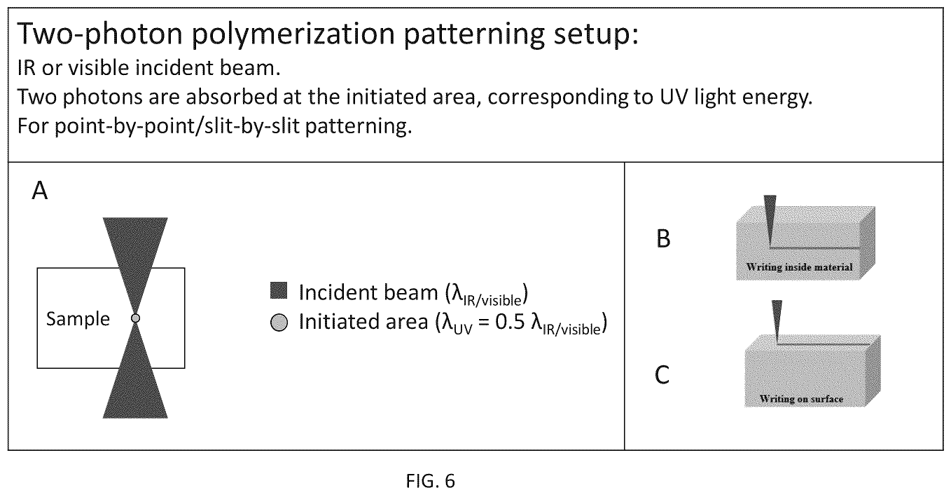

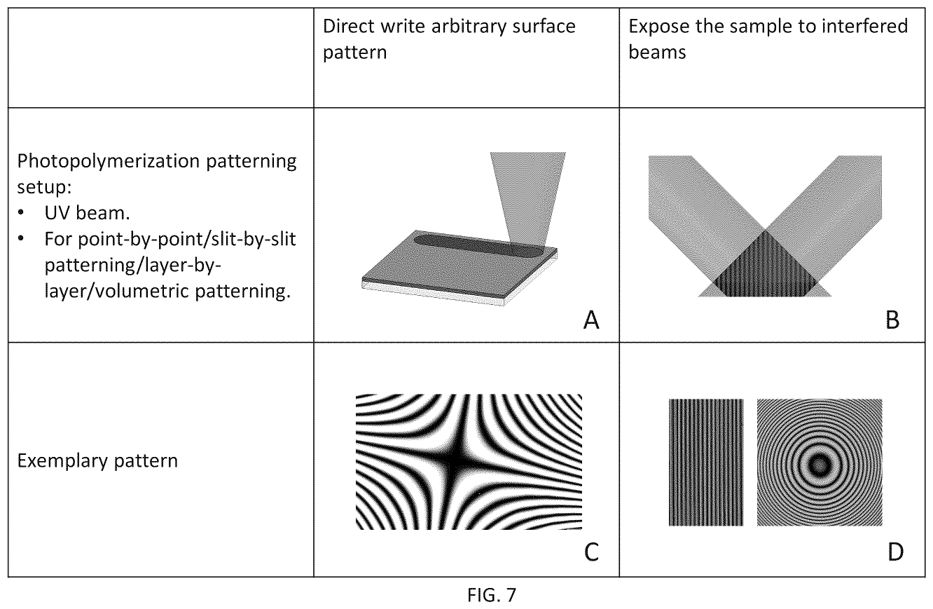

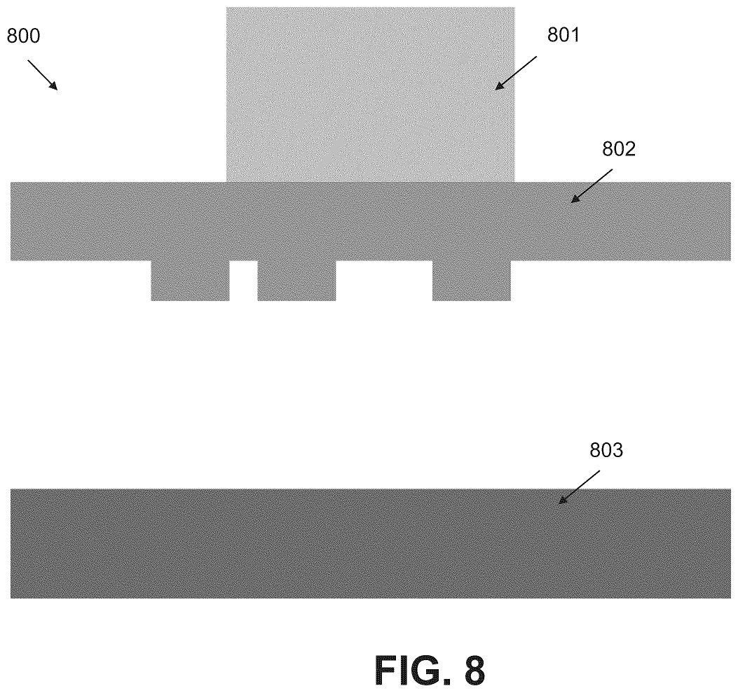

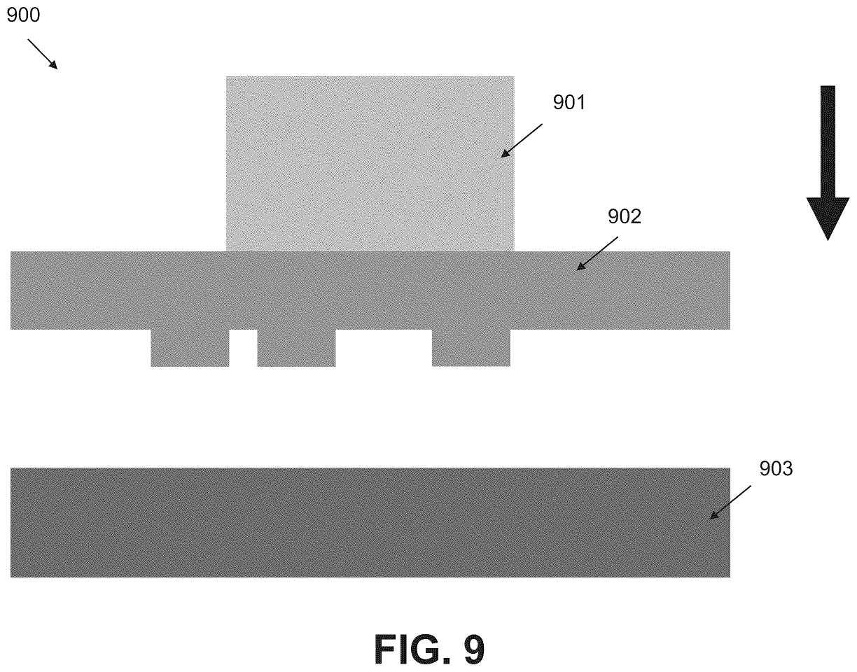

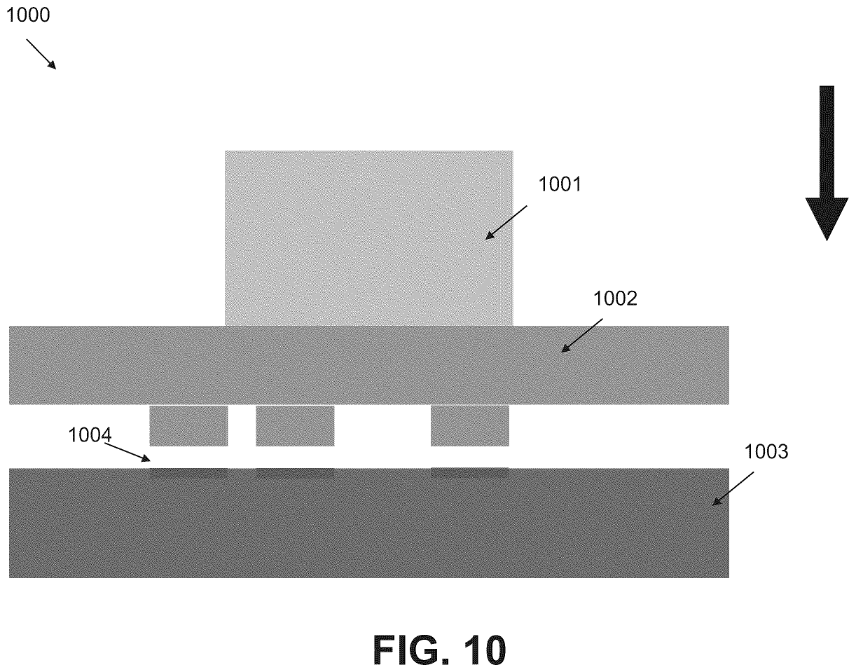

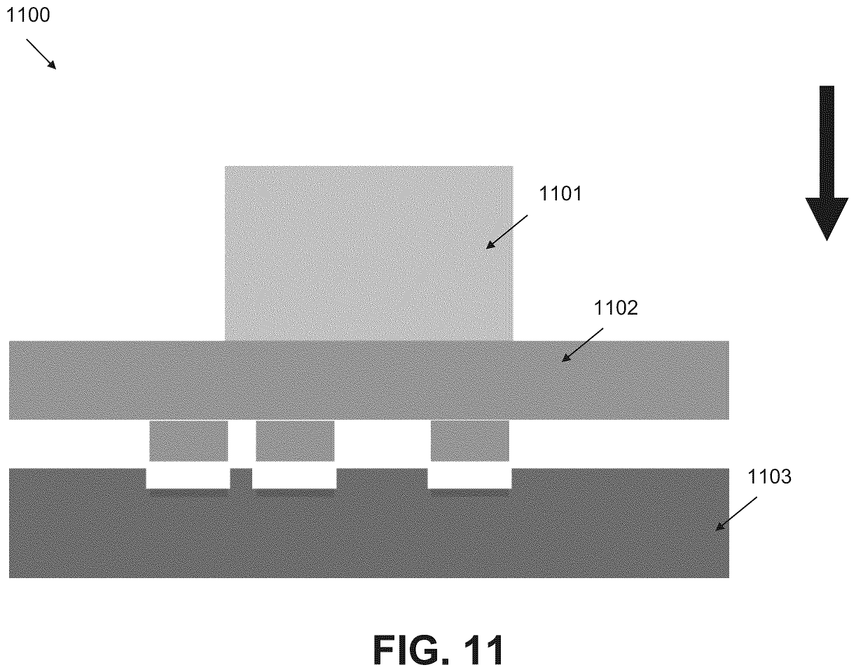

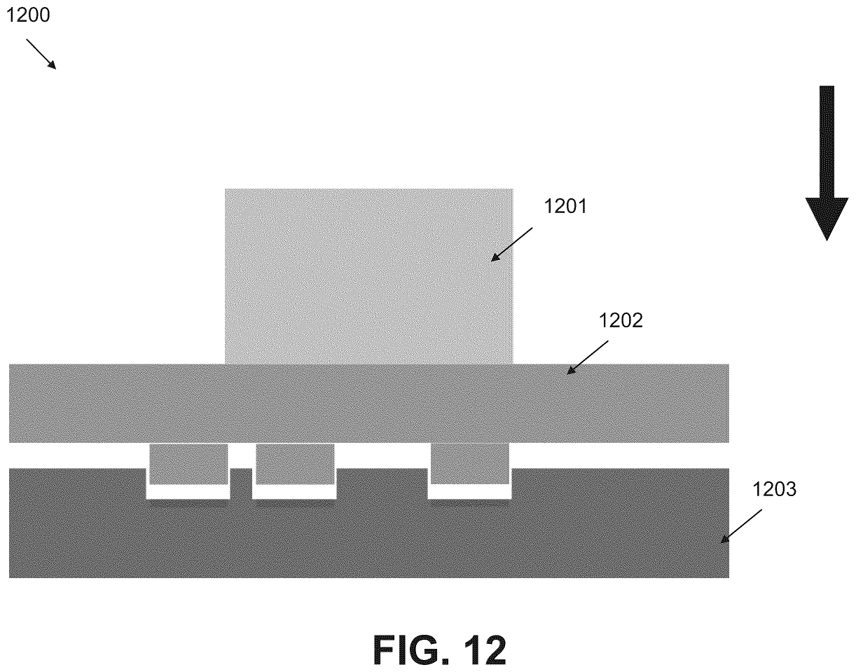

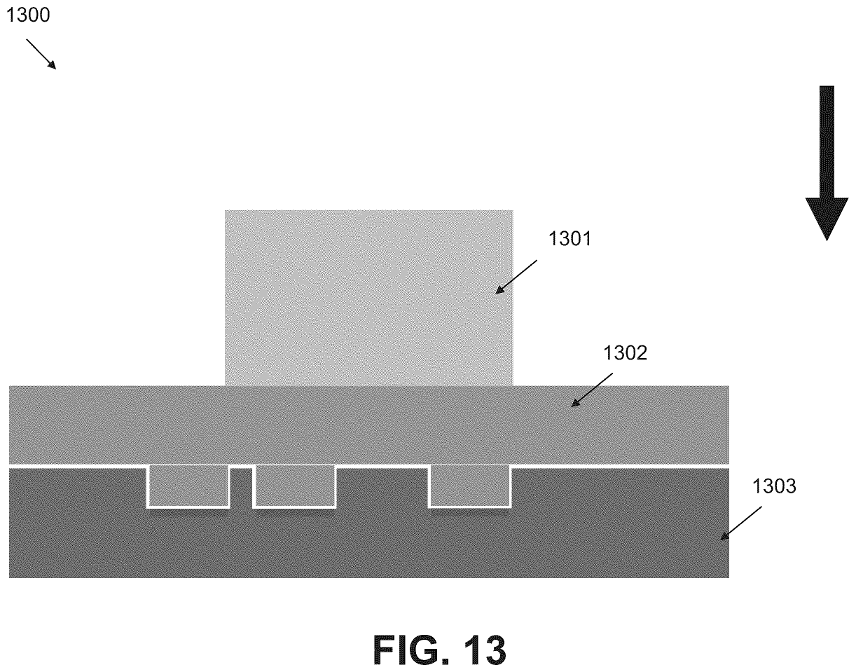

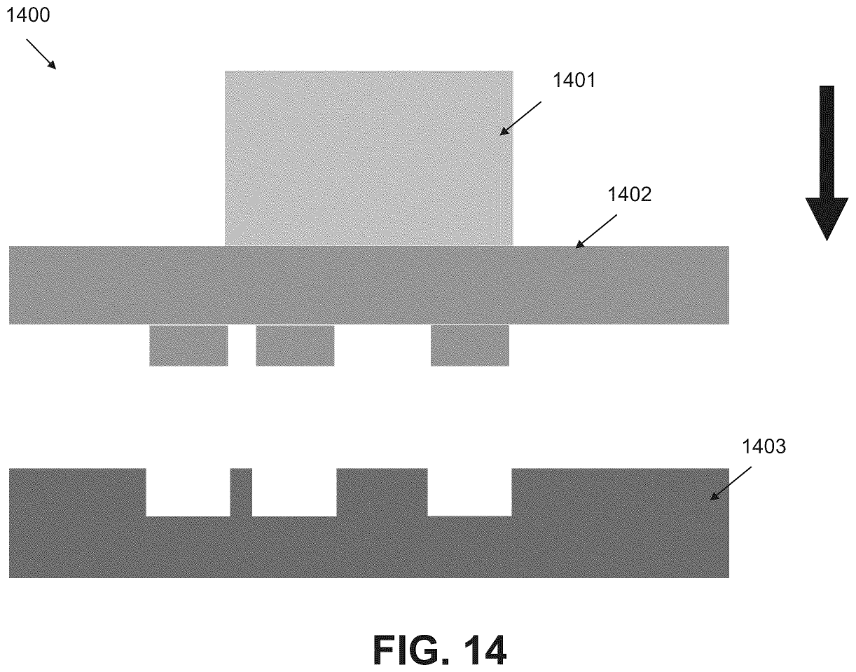

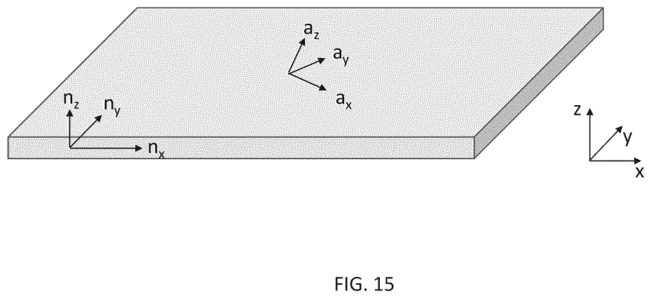

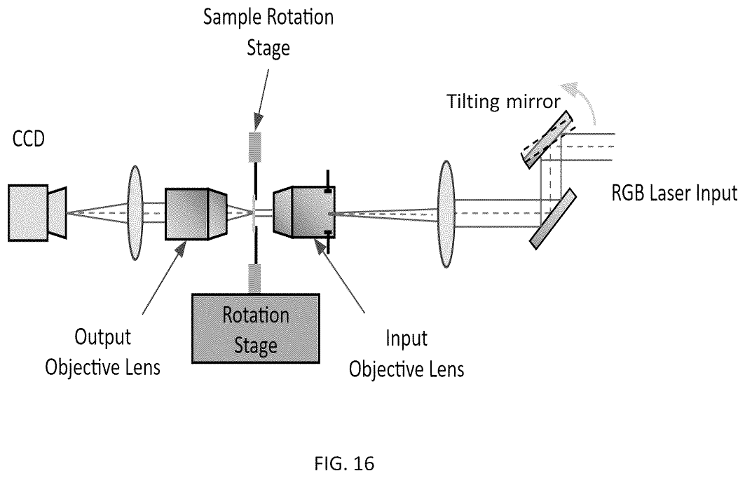

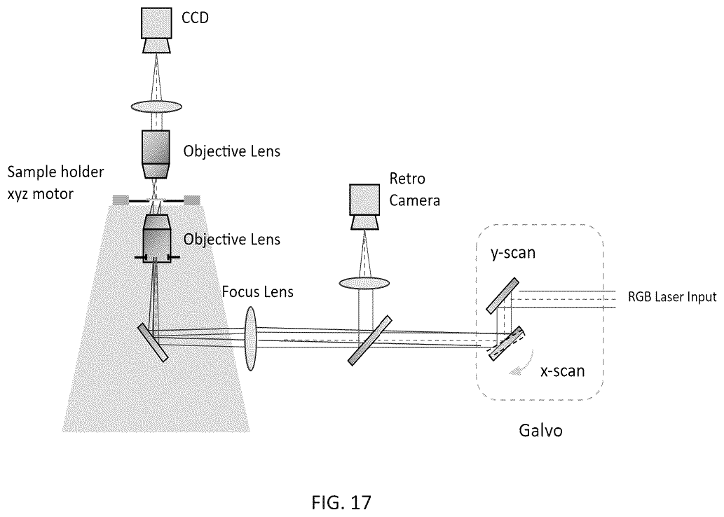

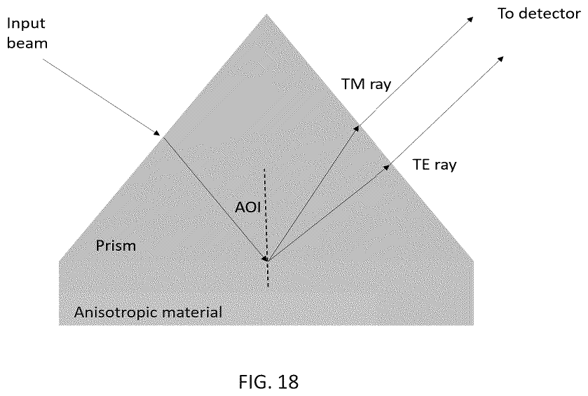

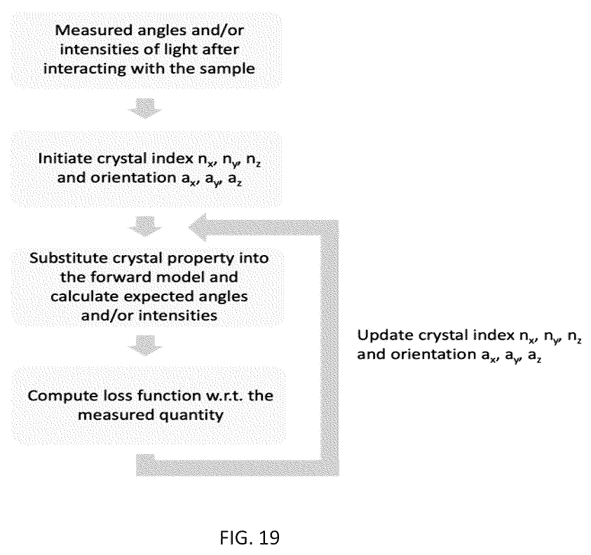

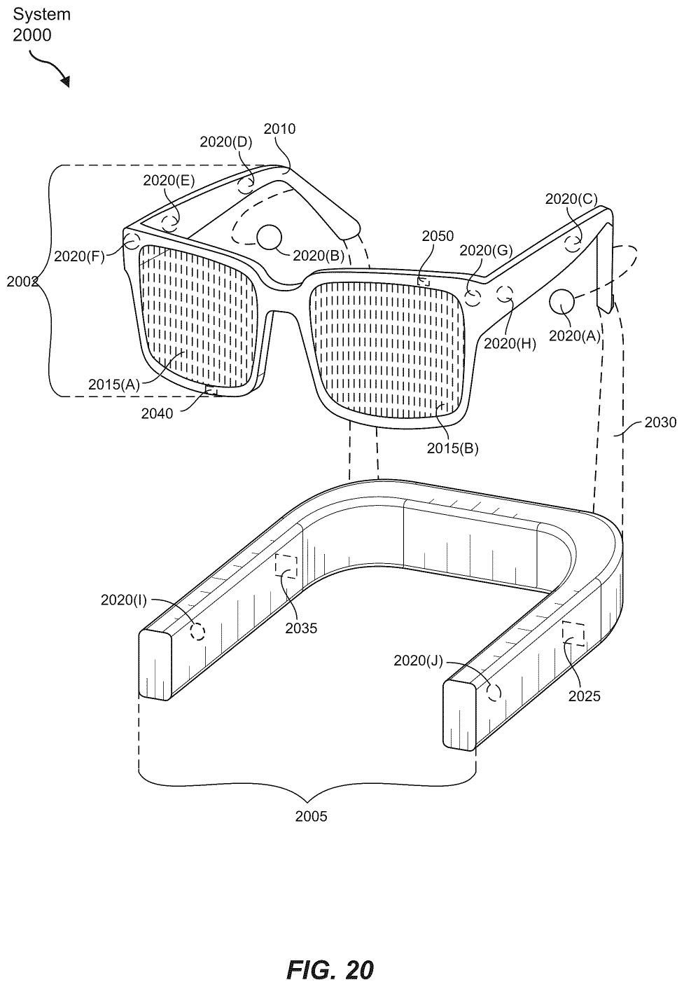

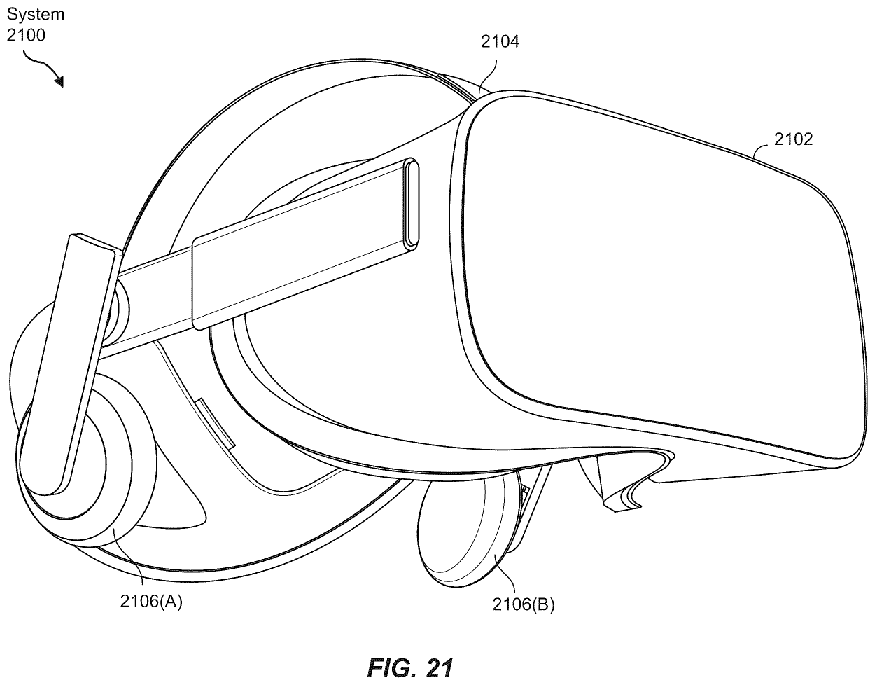

The following will provide, with reference to FIGS. 1-21, detailed descriptions of systems and methods for optical tracking. Detailed descriptions of computer-implemented methods for calibrating and/or training radar-based tracking systems will be provided in connection with FIG. 1. Detailed descriptions of corresponding example systems will also be provided in connection with FIG. 2. Detailed descriptions of an example reflector will be provided in connection with FIG. 3. Detailed descriptions of an example block diagram of a signal will be provided in connection with FIG. 4. Detailed descriptions of an example block diagram of a process for training a machine learning model will be provided in connection with FIG. 5. Detailed descriptions of example two-photon polymerization methods will be provided in connection with FIG. 6. Detailed descriptions of example bulk and surface photopolymerization will be provided in connection with FIG. 7. Detailed descriptions of an example embossing apparatus will be provided in connection with FIG. 8-9. Detailed descriptions of an example embossing apparatus and its impact on the organic solid crystal (OSC) material will be provided in connection with FIGS. 10-14. Detailed descriptions of example optical and crystallographic properties of OSC will be provided in connection with FIG. 15. Detailed descriptions of an example metrology apparatus and their methods operation will be provided in connection with FIG. 16-17. Detailed descriptions of an example technique for evaluating the crystallographic and optical properties of an optically anisotropic solid material will be provided in connection with FIG. 18. Detailed descriptions of an example algorithm for determining the crystallographic and optical properties of an optically anisotropic material will be provided in connection with FIG. 19. Detailed descriptions of exemplary augmented reality and virtual reality devices that may include the corresponding optical tracking methods will be provided in connection with FIGS. 20-21.

FIG. 1 is a flow diagram of an exemplary computer-implemented method 100 for calibrating and/or training radar-based tracking systems. The steps shown in FIG. 1 may be performed by any suitable computer-executable code and/or computing system, including the system(s) illustrated in FIG. 2. In one example, each of the steps shown in FIG. 1 may represent an algorithm whose structure includes and/or is represented by multiple sub-steps, examples of which will be provided in greater detail below. In some examples, the steps shown in FIG. 1 may be performed by a controller 202 and/or a radar component 204 (e.g., operating in and/or in connection with a user device 206, as depicted in FIG. 2, and/or a server). In these examples, the server may represent any type or form of backend computing device that may perform one or more functions directed at radar detection, artificial reality, and/or social networking.

User device 206 generally represents any type or form of computing device capable of reading computer-executable instructions. In some examples, user device 206 may represent an artificial reality device. In these examples, user device 206 generally represents any type or form of system, capable of reading computer-executable instructions, designed to provide an artificial reality experience to a user, such as one or more of the systems that will be described later in connection with FIGS. 20-21 and/or a computing device (e.g., a console and/or a mobile device) configured to operate in conjunction with one or more of the systems that will be described later in connection with FIGS. 20-21. In some examples, user device 206 may represent a smart phone and/or a tablet. Additional examples of user device 206 may include, without limitation, a laptop, a desktop, a wearable device, a personal digital assistant (PDA), etc.

In some examples, user device 206 may include one or more reflectors 208 (e.g., metallic reflectors), specifically designed and placed on user device 206 to broaden (e.g., focus) the field of view of a radar sensor (e.g., radar component 204), which may be otherwise limited by line of sight. Reflectors 208 can represent any type or form of reflective (e.g., metallic) surface or material. In some examples, the reflectors may include and/or represent full metal (e.g., sheet metal) and/or a structured metal surface. Reflectors 208 may enable radar component 204 to achieve sensing around obstacles and to collect more precise and accurate sensing data (e.g., enabling radar component 204 to track smaller movements and to track movement more precisely). Additionally, reflectors 208 may act as a radar component multiplier (e.g., enabling one radar component to detect multiple angles and locations).

Reflectors 208 may be positioned anywhere within user device 206 (e.g., within an AR device with radar-tracking capabilities). In one example, a reflector can be placed or printed on the inner or outer surface of user device 206 in a position that reflects radar waves from radar component 204 towards an object (e.g., a face or portion of a face) that would otherwise be inaccessible.

Reflectors 208 may have a wide variety of designs (e.g., a concave and/or convex metal surface). In some examples, reflectors 208 can divide and/or steer radar directions. In one embodiment, reflectors 208 may include multiple reflectors with different orientations and focal distances. In this embodiment, reflectors 208 may segment an outgoing radar beam into multiple beams (e.g., emitted in different directions). Additionally or alternatively, reflectors 208 may redirect return radar beams. In some examples, a position of radar component 204 relative to reflectors 208 may be selected to achieve a desired signal strength (e.g., to increase signal strength) and/or response time segmenting. FIG. 3 depicts reflected radar beam access and a radar field of view that are altered based on a placement of a reflector 300 relative to radar component 204.

Returning to FIG. 1, at step 110, one or more of the systems described herein may cause a radar component to emit one or more radar signals. For example, as illustrated in FIG. 2, controller 202 may cause radar component 204 to emit a radar signal 210.

Controller 202 may represent any type or form of hardware device and/or software program configured to control a radar component. In some examples, controller 202 may operate within an artificial reality context. In one such example, controller 202 may represent a handheld input device that enables a user to manipulate (e.g., interact with) an artificial (e.g., virtual) environment. In other examples, controller 202 may represent and/or operate as part of a user device and/or a server.

Radar component 204 may represent any component of a radar tracking system. In some examples, radar component 204 may represent a radio detection and ranging (e.g., radar) sensor that uses radar signals (e.g., radio waves) to detect objects in its surrounding environment (e.g., by transmitting a radio signal to an object and then detecting the reflected signal bouncing back from the object). In some examples, radar component 204 may represent a close-range radar sensor (e.g., operating at a close range such as 1-5 mW). In some examples, radar component 204 may emit radar signals that penetrate occlusive objects such as hair, masks, gloves, etc. Additionally, radar component 204 may be able to collect radar data (e.g., a return radar signal) in variable lighting conditions.

In one embodiment, radar component 204 may include a transmitter 212, configured to emit radar signals (e.g., radar signal 210), an antenna 214, configured to transmit the radar signals onto objects in the surrounding environment and receive the radar signals that return (e.g., bounce back) from the objects, and a receiver 216, configured to detect the return signals and/or process the return signals to extract information (e.g., features of an object) based on the return signal (e.g., by measuring displacement of return signals as will be described in greater detail below in connection with step 120).

Radar component 204 may operate within a variety of contexts and may be communicatively coupled to (e.g., embedded within) a variety of devices. In some examples, radar component 204 may operate as part of an artificial reality device (e.g., a device with one or more of the features described above in connection with FIG. 2, FIG. 20 and FIG. 21). In some examples, radar component 204 may be configured to emit radar signal 210 onto an object 218 to determine information about object 218 (as will be described in greater detail in connection with step 120). Object 218 may refer to any type or form of object onto which radar signal 210 may be emitted. In some examples (e.g., in which controller 202 and/or radar component 204 operate as part of a user-tracking system such as a face-tracking system), object 218 may represent a user, a user face, and/or a user body (e.g., body part).

At step 120, one or more of the systems described herein may analyze one or more return signals (e.g., received by a receiver of the radar component in response to a transmitter of the radar component having emitted the radar signals). For example, as illustrated in FIG. 2, controller 202 may cause radar component 204 (e.g., receiver 216 of radar component 204) to analyze a return signal 220. In this example, return signal 220 may represent radar signal 210 that has returned (i.e., after reflecting off object 218) and been received (e.g., by receiver 216) after being emitted onto object 218 (e.g., by transmitter 212) and reflected off object 218 (e.g., back to receiver 216), as depicted in FIG. 4.

Radar component 204 may analyze return signal 220 to generate a variety of data. In some examples, radar component 204 may analyze return signal 220 to generate data identifying one or more features of object 218 (e.g., using a feature extraction process). In some examples, a feature may represent an identification of object 218 (e.g., a face or a hand) and/or a component of object 218 (e.g., an eyebrow or a finger). Additionally or alternatively, a feature may represent a movement of object 218 and/or a component of object 218 (e.g., a raised eyebrow, a lip pinch, a finger snap, etc.).

Radar component 204 (e.g., receiver 216 of radar component 204) may analyze return signal 220 in a variety of ways. In some examples, radar component 204 may rely on a trained machine learning model (e.g., trained model 222) to analyze return signal 220. In one example, the machine learning model may be configured to learn (e.g., using user-specific camera data) to customize facial tracking for a particular user (e.g., the user associated with user device 206).

The machine learning model may be trained in a variety of ways. In some examples, the machine learning model may be trained on concurrently collected radar data (e.g., radar features extracted from radar frames) and camera images (e.g., landmark coordinates such as face coordinates extracted from camera frames), as shown in machine learning training process 500 in FIG. 5. As shown in FIG. 5, in some examples, the machine learning model may be evaluated for accuracy (e.g., using a Root Mean Square Error (RMSE) technique) and adjustments (e.g., using a training gradient) may be applied.

The term “landmark” may refer to any type or form of specific point or feature that can be used as a reference. In some examples, a landmark may refer to a facial landmark. Examples of facial landmarks may include, without limitation, inner or outer corners of the eyes, the tip of the nose, the high point of the eyebrows, the corners of the mouth, one or more points along the jawline, etc.

The disclosed machine learning model may be trained using calibration expressions for individual users. Examples of calibration expressions may include a neutral estimator. The neutral estimator may represent an estimate of a neutral pose for a subject (e.g., object 218) and the machine learning model may be trained to estimate the neutral pose and/or to predict displacement from the neutral pose. In examples in which object 218 represents a user's face, the neutral estimator may represent an estimate of the face in a neutral position (e.g., a set of parameters corresponding to a pose of the face in the neutral position). Using a neutral estimator allows the machine learning model to identify features based on landmark displacement (relative to the neutral estimate) that is determined based on changes in radar signal (e.g., instead of requiring the model to identify features based on absolute landmark position). Other calibration expressions may include jaw open, smiling, puckering, eyebrow lifting, and asymmetric facial movements.

The calibration expressions may be generated in real-time and/or during a calibration process. In some examples, the disclosed systems may complete a calibration process by digitally prompting a user (e.g., the user of user device 206) to (1) hold a neutral expression for a designated period (e.g., 5 seconds) and (2) make random expressions for an additional designated period (e.g., 25 seconds). During the two designated periods, the disclosed systems may collect (1) camera images (e.g., captured using a camera of user device 206) and (2) radar data (e.g., emitted and received via radar component 204) and apply this data to train the machine learning model. In one embodiment, the disclosed systems (e.g., the machine learning model) may compare a feature predicted by the machine learning model (e.g., an eyebrow raise or an eyebrow lower) with a ground truth (based on a camera image). In some instances, a ground truth may not be available (e.g., in instances in which the feature involves upper face tracking and an image of the upper face is unavailable due to occlusion of the upper face).

Radar component 204 may apply a variety of data to the machine learning model (e.g., to determine landmark displacement). In some examples, rather than applying raw signals (i.e., return signal 220) to the machine learning model, radar component 204 may apply a representation of radar data (e.g., extracted from return signal 220) that encodes phase changes (e.g., shifts in the position of return signal 220 relative to radar signal 210) using a complex (e.g., multi-layered) representation. In one such example, the representation may encode phase (e.g., without conventional wrapping issues) using scaled real-imaginary parts of Fourier coefficients. In some examples, the radar data in the representation may have been normalized (e.g., calibrated) using the neutral estimator described above.

Radar component 204 may measure (e.g., extract) a variety of data from return signal 220. Examples of such data include, without limitation, measures of depth (e.g., the distance between radar component 204 and object 218), displacement (e.g., a change in position of object 218 relative to a neutral estimator and/or a change in radar signal 210 relative to return signal 220), and/or velocity (e.g., a speed and/or direction of motion of object 218). In contrast to the physical parameters (e.g., optical contrast) measured by a camera, the return signal data extracted by radar component 204 may not be human-interpretable. Radar component 204 may translate such data into usable data in a variety of ways. For example, radar component 204 may (e.g., using the machine learning techniques described above) use displacement information to translate radar data into landmarks (e.g., facial landmarks in embodiments in which the radar tracking system identifies physical user features) and/or changes in landmarks.

While the examples above focus on facial recognition, the disclosed radar tracking system may use radar data (e.g., collected via radar component 204 using one or more of the techniques described above) to detect (e.g., track) a variety of information. In one example, radar data may be used to detect a user's heartbeat (e.g., based on body movement and/or pulse information extracted from displacement detected at the user's temple). As another example, the radar data may be used to detect respiration rates (e.g., based on chest movements detected based on displacement detected at a user's chest). In some examples, the disclosed radar tracking system may be implemented in an electronic health-monitoring device (e.g., radar component 204 may be mounted in eyewear for health sensing).

While the examples discussed in connection with FIG. 1 focus on an embodiment in which a controller causes radar component 204 to emit and/or analyze radar signals, radar component 204 may emit and/or analyze radar signals in response to any type or form of input from any computerized device.

A two-photon polymerization method for processing a liquid crystal material is illustrated schematically in FIG. 6. Referring to FIG. 6A, a near-IR or visible beam may provide minimal surface interaction and amplified penetration with a photopolymerizable material. At the focal point of the beam, two photons are absorbed by the photopolymerizable material, and the corresponding UV light energy initiates local polymerization. A two-photon polymerization method may be used to code an interior volume or near surface region of a photopolymerizable material, as depicted in FIGS. 6B and 6C, respectively.

Referring to FIG. 7, a comparative photopolymerization method may be used to direct write arbitrary surface pattern by moving the sample stage (FIG. 7A), or to expose the photopolymerizable material to interfered beams to record polarization fringes or the phase profile of lenses (FIG. 7B). Exemplary patterned polarization profile are depicted in FIGS. 7C and 7D, respectively.

In some embodiments, a method to characterize a diffractive waveguide may include capturing full-aperture measurements for characterization of spatial non-uniformities across the waveguide clear aperture. Full-aperture measurements described herein may include a single image or multiple images of an entire area of a waveguide material and/or elements of the waveguide structure. In some methods, the elements of the waveguide structure may include but are not limited to, e.g., a liquid crystal polymer with characteristics of inherent polarization control, polarization volume holograms, surface relief gratings, and volume Bragg gratings. The characteristics of the waveguide material may enable measurement of a polarization response or measurement of selected matrices to back calculate (e.g., polarization ray trace calculation) the elements of the waveguide structure. The polarization response from the waveguide material may enable the measurement of the polarization properties at various wavelengths on a pixel-to-pixel scale. In further embodiments, the full-aperture images may be captured via a camera (e.g., polarization camera, multispectral full-Stokes polarization camera) where the images may be represented as multi-dimensioned data. In some examples, the full-aperture images may be captured within the visible light spectrum, infrared spectrum, or ultraviolet spectrum.

In some embodiments, the method to characterize a diffractive waveguide may include measuring an input ray to the waveguide. The polarization state of the input ray may be dynamically controlled prior to entering the waveguide in use. In further embodiments, the input rays may diffract into multiple rays within the path of the waveguide, where the rays may replicate in separate diffraction orders. In some methods, the replicated rays may be measured at the waveguide output using the polarization camera. The waveguide may be operational as a modulator to transmit the output rays to the polarization camera. In further embodiments, the polarization camera may capture the outputted rays and conduct data analysis via polarization ray trace. With the combination of simulated tools, the waveguide structure may be calculated from the measured output rays. According to some embodiments, a matrix inversion method may be used during analysis of the output rays to back calculate the waveguide structure.

In some embodiments, the method to characterize a diffractive waveguide may include an optical system. The optical system may be used to generate multidimensional rays of data with selective control of the polarization state in terms of wavelength across the waveguide structure in use. According to some embodiments, the optical system may include a light source (e.g. polarization state generator), a diffractive optical element (DOE) coupled to a substrate, and an analyzer (e.g., polarization state analyzer). The light source may transmit rays to the waveguide and generate any polarization state as a function of wavelength. The transmitted rays from the light source may enter a diffractive optical element (DOE), where the rays are diffracted and replicated within the path of the waveguide. In some embodiments, the DOE is coupled to a substrate that may eliminate replicated pupils that pass through the DOE. In this manner, the substrate may filter the extraneous rays that pass through the DOE to result in a single ray that may be viable for use in a simulation. In some methods, the diffracted rays interact with the substrate to spread in various angular directions or diffraction orders (e.g., T-0, T-1, T-2, etc.) for analysis via the polarization analyzer. The polarization analyzer may analyze the polarization state of transmitted rays as a function of wavelength. In some examples, the polarization analyzer may be used in combination with a polarization camera to measure the selected zeroth order (T-0) of the transmitted rays and capture the polarization data as a function of wavelength. In further embodiments, the optical system may include a tilted optical wedge coupled to the DOE that may separate the transmitted rays into separate diffraction orders.

In some embodiments, the method to characterize a diffractive waveguide may include a full aperture optical system. The full aperture optical system may include a dove-prism, a polarization volume hologram (PVH), a collimated light source, and a polarization analyzer. In some methods, the dove-prism may enable the full aperture optical system to selectively control the diffraction order transmission of input rays with free-pupil implications from all the diffraction orders. In some methods, the dove prism may be kept at an angle that is equivalent to the angle of incidence of the transmitted ray input to the PVH. The optical system configuration may enable the input transmit ray to be incident normally on the surface of the dove prism. In some examples, the tilt angle of the dove prism may be variably altered for diffraction orders of the transmit rays (e.g., R-1 and T-1) to transmit normally to the surface of the PVH. The surface of the PVH may include a grating pitch constructed from a material including but not limited to an organic material, a high-index non-organic material, a photo-polymer material etc . . . . In this manner, the R-1 and T-1 transmit rays may exit a second side of the prism un-refracted. In further examples, the diffraction order of the input transmits rays (e.g., R-0) may be symmetrical around the prism surface normal of a PVH coating (e.g. reflective PVH, transmissive PVH, etc.) and exit the other side of the prism un-refracted. Additionally, the optical system may include R-1 order measuring that may measure each of the diffractive orders as a function of wavelength and polarization sensitivity. In further examples, the polarization camera may capture an image of any transmit ray diffraction order by moving in a spherical motion. In this manner the polarization camera may capture full-aperture image data of transmit rays that may include, e.g., a spectral dimension, a spatial dimension (e.g., X-Y dimension), and a polarization dimension for measuring the waveguide structure. In some embodiments, the full aperture optical system may include a polarization analyzer used in combination with the polarization camera to add the polarization dimension data to the captured image. Further, a filter (spectral filter wheel, color filter wheel, etc.) may be added to the polarization camera and/or polarization analyzer to add multi-spectral elements and hyper-spectral elements to the captured image. According to some embodiments, the full aperture optical system may include an integrated sphere on one end of the system.

In some embodiments, the method to characterize a diffractive waveguide may include a scanning-based imaging method (e.g., point-scanning imaging, line-scanning imaging, etc.). The scanning method may include a tunable light source, a PVH coated on a substrate, a stage (e.g., XY stage), and polarization analyzer. In some methods, the tunable light source may scan a waveguide structure placed on the stage via incident light. The incident light may probe the waveguide structure and enter the aperture of the polarization analyzer to measure the polarization data. In this manner, the scanning method may build a 2-D raster map of the waveguide structure.

FIG. 8 is a diagram of an embossing apparatus, in accordance with some embodiments. The embossing apparatus 800 may include a temperature and motion controller 801 (i.e., TMC), a thermal device 802, and a substrate 803. The TMC 801 may be configured to thermal device 802 to enable dynamic motion and temperature control to thermal device 802. As discussed below in conjunction with FIG. 9-14, thermal device 802 may include at least one source of assembly to generate a patterned source of thermal energy to transfer a pattern onto substrate 803. In some embodiments, the patterned source of thermal energy may include, e.g., a patterned template, where the pattern is variably interchangeable to transfer a desired pattern. Substrate 803 may include, e.g., a crystalline material, organic solid crystal (OCS) material, thermally stable material, conductive material that can be sublimated.

FIG. 9 is a perspective view 900 of embossing apparatus 800, in accordance with some embodiments of the present disclosure. Embossing apparatus 900 may include at least a TMC 901, a thermal device 902, a substrate 903, and thermal transmitter 904A-904C. In some embodiments, embossing apparatus 900 may operate with selective processing conditions to create a pattern with a variable 3-D structure on substrate 903 with thermal device 902.

According to certain embodiments, the processing conditions will be selective in a manner that changes the surface structure of substrate 903 through sublimation without compromising the crystallinity of substrate 903 that is retained. In contrast to comparative patterning techniques that use melting or molding, disclosed are surface modification methods that include the localized sublimation of an organic solid crystal. In some methods, selected portions of an OSC material are heated and removed by sublimation to the exclusion of unselected portions.

The processing conditions for effective sublimation of at least a portion of substrate 903 may include maintaining temperature and pressure values below a triple point (e.g., higher than 100° C., lower than 1 Torr, etc.) enabling at least a portion of the substrate 903 to be removed to the gas phase thus leaving solid material that is not encumbered by an amorphous phase, and thus retaining the optical properties of substrate 903. Maintaining the pressure values below the triple point may enable substrate 903 to sublimate rather than melt when heated.

In some examples, embossing apparatus 900 may operate under a sufficient vacuum (e.g., vacuum chamber) for successful sublimation of substrate 903. According to some embodiments, embossing apparatus 900 accomplishes sublimation of substrate 903 surface structure through dynamic motion of the TMC 901 to move thermal device 902 towards the proximity of substrate 903 or vice versa. TMC 901 may heat thermal device 902 to initiate the transfer of thermal energy to the surface of substrate 903 to induce sublimation for a resulting pattern. In some embodiments, TMC 901 may variably control the temperature of thermal device 902 and/or substrate 903.

FIG. 10 is perspective view of embossing apparatus 1000 locally heating portions of substrate 1003 with thermal device 1002. In some embodiments, embossing apparatus 1000 may locally heat portions of substrate 1003 via radiation from thermal device 1002. The locally heated portions of substrate 1003 may include portions of the surface material. In some embodiments, thermal device 1002 may include a nano-patterned template capable of transferring the desired pattern onto the surface of substrate 1003. When the heated thermal device approaches the proximity of substrate 1003, the surface material may enter the gas phase via sublimation and be evacuated from the processing area. In some methods, a microscale gap 1004 may occupy a space between thermal device 1002 and substrate 1003. Microscale gap 1004 may eliminate any physical contact during the transfer of thermal energy from thermal device 902.

FIG. 11 is a perspective view of embossing apparatus 1100 removing locally heated portions of substrate 1103 surface via sublimation to form a patterned surface. In some embodiments, the locally heated portions that are evaporated from substrate 1103 via heated thermal device 1102 may be evacuated from the processing area. Thermal device 1102 may have mechanisms for effective evacuation of the surface material including but not limited to, e.g., pores, vias, vacuum chamber with an active flow of carrier gas, micro holes, channels, etc. Turning to FIG. 12, controlling a depth of features within the patterned surface is shown. The desired pattern transferred to the surface of the surface material may vary in depth depending on the depth maintained by the TMC. In some embodiments, the thermal device may include a flat surface template to flatten a rough OSC material surface. For example, the rough OSC material surface may be flattened and smoothed via the flat surface template, which may contribute to the formation of optical structures.

FIG. 13 is a perspective view of embossing apparatus 1300 maintaining a constant distance between heated thermal device 1302 and the patterned surface material of substrate 1303. Turning to FIG. 14, TMC 1401 may withdraw the patterned template of thermal device 1402 from the patterned surface of substrate 1403.

Referring to FIG. 15, an optically anisotropic material such as an organic solid crystal may be characterized by its principal refractive indices (nₓ, n_{y}, n_{z}) and its principal symmetry axes (aₓ, a_{y}, a_{z}) relative to a reference coordinate (x, y, z).

Referring to FIG. 16, shown is a schematic view of an example metrology system for evaluating the optical parameters (nₓ, n_{y}, n_{z}, aₓ, a_{y}, a_{z}) of an optically anisotropic material. A sample may be mounted to a pair of rotation stages that are configured to control the orientation of the sample with respect to an incident beam of light such as laser light. For example, a lower stage rotation may vary the laser incident angle on the sample and an upper stage rotation may orient the sample through 3600 with respect to the optical axis of the laser. A tilting mirror may be adapted to alter the angle of incidence of the light incident upon the sample.

According to further embodiments, a laser microscopy OSC index metrology system for evaluating the optical parameters (nₓ, n_{y}, n_{z}, aₓ, a_{y}, a_{z}) of an optically anisotropic material is shown schematically in FIG. 17. A sample may be fixed in a tip tilt holder to locate the normal incident angle. A Galvo system may be configured to guide a laser beam over the sample and provide a variable incident angle with high precision and repeatability.

Referring to FIG. 18, shown schematically is a further metrology technique using a prism coupler in conjunction with a critical angle phenomenon. For a given angle of incidence (AOI) an input beam may be split into two orthogonal polarizations (i.e., TM and TE) due to interaction with the anisotropic material. From the split rays, which represent different critical angles, refractive index and crystal orientation information may be derived.

In conjunction with the systems of any of FIGS. 16-18, optical information, including the angle and polarization state of input and output light, may be determined and fed to an algorithm that evaluates the optical parameters of the sample in a manner that rationalizes the transformation of input light character to output light character.

An example workflow for crystal index and orientation retrieval is outlined in the flowchart of FIG. 19. The angles and/or intensities of light before and after interacting with a sample are measured and input into an algorithm. Index and crystal orientation parameters are selected by the model and used to determine expected angles and/or intensities, which are evaluated in terms of the measured angles and/or intensities to generate an error metric, i.e., loss function. The algorithm updates the selected parameters and repeats the evaluation so as to minimize the error metric and obtain a crystal index and orientation mapping of the sample.

Embodiments of the present disclosure may include or be implemented in conjunction with various types of artificial-reality systems. Artificial reality is a form of reality that has been adjusted in some manner before presentation to a user, which may include, for example, a virtual reality, an augmented reality, a mixed reality, a hybrid reality, or some combination and/or derivative thereof. Artificial-reality content may include completely computer-generated content or computer-generated content combined with captured (e.g., real-world) content. The artificial-reality content may include video, audio, haptic feedback, or some combination thereof, any of which may be presented in a single channel or in multiple channels (such as stereo video that produces a three-dimensional (3D) effect to the viewer). Additionally, in some embodiments, artificial reality may also be associated with applications, products, accessories, services, or some combination thereof, that are used to, for example, create content in an artificial reality and/or are otherwise used in (e.g., to perform activities in) an artificial reality.

Artificial-reality systems may be implemented in a variety of different form factors and configurations. Some artificial-reality-systems may be designed to work without near-eye displays (NEDs). Other artificial-reality systems may include an NED that also provides visibility into the real world (such as, e.g., augmented-reality system 2000 in FIG. 20) or that visually immerses a user in an artificial reality (such as, e.g., virtual-reality system 2100 in FIG. 21). While some artificial-reality devices may be self-contained systems, other artificial-reality devices may communicate and/or coordinate with external devices to provide an artificial-reality experience to a user. Examples of such external devices include handheld controllers, mobile devices, desktop computers, devices worn by a user, devices worn by one or more other users, and/or any other suitable external system.

Turning to FIG. 20, augmented-reality system 2000 may include an eyewear device 2002 with a frame 2010 configured to hold a left display device 2015(A) and a right display device 2015(B) in front of a user's eyes. Display devices 2015(A) and 2015(B) may act together or independently to present an image or series of images to a user. While augmented-reality system 2000 includes two displays, embodiments of this disclosure may be implemented in augmented-reality systems with a single NED or more than two NEDs.

In some embodiments, augmented-reality system 2000 may include one or more sensors, such as sensor 2040. Sensor 2040 may generate measurement signals in response to motion of augmented-reality system 2000 and may be located on substantially any portion of frame 2010. Sensor 2040 may represent one or more of a variety of different sensing mechanisms, such as a position sensor, an inertial measurement unit (IMU), a depth camera assembly, a structured light emitter and/or detector, or any combination thereof. In some embodiments, augmented-reality system 2000 may or may not include sensor 2040 or may include more than one sensor. In embodiments in which sensor 2040 includes an IMU, the IMU may generate calibration data based on measurement signals from sensor 2040. Examples of sensor 2040 may include, without limitation, accelerometers, gyroscopes, magnetometers, other suitable types of sensors that detect motion, sensors used for error correction of the IMU, or some combination thereof.

In some examples, augmented-reality system 2000 may also include a microphone array with a plurality of acoustic transducers 2020(A)-2020(J), referred to collectively as acoustic transducers 2020. Acoustic transducers 2020 may represent transducers that detect air pressure variations induced by sound waves. Each acoustic transducer 2020 may be configured to detect sound and convert the detected sound into an electronic format (e.g., an analog or digital format). The microphone array in FIG. 20 may include, for example, ten acoustic transducers: 2020(A) and 2020(B), which may be designed to be placed inside a corresponding ear of the user, acoustic transducers 2020(C), 2020(D), 2020(E), 2020(F), 2020(G), and 2020(H), which may be positioned at various locations on frame 2010, and/or acoustic transducers 2020(I) and 2020(J), which may be positioned on a corresponding neckband 2005.

In some embodiments, one or more of acoustic transducers 2020(A)-(J) may be used as output transducers (e.g., speakers). For example, acoustic transducers 2020(A) and/or 2020(B) may be earbuds or any other suitable type of headphone or speaker.

The configuration of acoustic transducers 2020 of the microphone array may vary. While augmented-reality system 2000 is shown in FIG. 20 as having ten acoustic transducers 2020, the number of acoustic transducers 2020 may be greater or less than ten. In some embodiments, using higher numbers of acoustic transducers 2020 may increase the amount of audio information collected and/or the sensitivity and accuracy of the audio information. In contrast, using a lower number of acoustic transducers 2020 may decrease the computing power required by an associated controller 2050 to process the collected audio information. In addition, the position of each acoustic transducer 2020 of the microphone array may vary. For example, the position of an acoustic transducer 2020 may include a defined position on the user, a defined coordinate on frame 2010, an orientation associated with each acoustic transducer 2020, or some combination thereof.

Acoustic transducers 2020(A) and 2020(B) may be positioned on different parts of the user's ear, such as behind the pinna, behind the tragus, and/or within the auricle or fossa. Or, there may be additional acoustic transducers 2020 on or surrounding the ear in addition to acoustic transducers 2020 inside the ear canal. Having an acoustic transducer 2020 positioned next to an ear canal of a user may enable the microphone array to collect information on how sounds arrive at the ear canal. By positioning at least two of acoustic transducers 2020 on either side of a user's head (e.g., as binaural microphones), augmented-reality device 2000 may simulate binaural hearing and capture a 3D stereo sound field around about a user's head. In some embodiments, acoustic transducers 2020(A) and 2020(B) may be connected to augmented-reality system 2000 via a wired connection 2030, and in other embodiments acoustic transducers 2020(A) and 2020(B) may be connected to augmented-reality system 2000 via a wireless connection (e.g., a BLUETOOTH connection). In still other embodiments, acoustic transducers 2020(A) and 2020(B) may not be used at all in conjunction with augmented-reality system 2000.

Acoustic transducers 2020 on frame 2010 may be positioned in a variety of different ways, including along the length of the temples, across the bridge, above or below display devices 2015(A) and 2015(B), or some combination thereof. Acoustic transducers 2020 may also be oriented such that the microphone array is able to detect sounds in a wide range of directions surrounding the user wearing the augmented-reality system 2000. In some embodiments, an optimization process may be performed during manufacturing of augmented-reality system 2000 to determine relative positioning of each acoustic transducer 2020 in the microphone array.

In some examples, augmented-reality system 2000 may include or be connected to an external device (e.g., a paired device), such as neckband 2005. Neckband 2005 generally represents any type or form of paired device. Thus, the following discussion of neckband 2005 may also apply to various other paired devices, such as charging cases, smart watches, smart phones, wrist bands, other wearable devices, hand-held controllers, tablet computers, laptop computers, other external compute devices, etc.

As shown, neckband 2005 may be coupled to eyewear device 2002 via one or more connectors. The connectors may be wired or wireless and may include electrical and/or non-electrical (e.g., structural) components. In some cases, eyewear device 2002 and neckband 2005 may operate independently without any wired or wireless connection between them. While FIG. 20 illustrates the components of eyewear device 2002 and neckband 2005 in example locations on eyewear device 2002 and neckband 2005, the components may be located elsewhere and/or distributed differently on eyewear device 2002 and/or neckband 2005. In some embodiments, the components of eyewear device 2002 and neckband 2005 may be located on one or more additional peripheral devices paired with eyewear device 2002, neckband 2005, or some combination thereof.

Pairing external devices, such as neckband 2005, with augmented-reality eyewear devices may enable the eyewear devices to achieve the form factor of a pair of glasses while still providing sufficient battery and computation power for expanded capabilities. Some or all of the battery power, computational resources, and/or additional features of augmented-reality system 2000 may be provided by a paired device or shared between a paired device and an eyewear device, thus reducing the weight, heat profile, and form factor of the eyewear device overall while still retaining desired functionality. For example, neckband 2005 may allow components that would otherwise be included on an eyewear device to be included in neckband 2005 since users may tolerate a heavier weight load on their shoulders than they would tolerate on their heads. Neckband 2005 may also have a larger surface area over which to diffuse and disperse heat to the ambient environment. Thus, neckband 2005 may allow for greater battery and computation capacity than might otherwise have been possible on a stand-alone eyewear device. Since weight carried in neckband 2005 may be less invasive to a user than weight carried in eyewear device 2002, a user may tolerate wearing a lighter eyewear device and carrying or wearing the paired device for greater lengths of time than a user would tolerate wearing a heavy standalone eyewear device, thereby enabling users to more fully incorporate artificial-reality environments into their day-to-day activities.

Neckband 2005 may be communicatively coupled with eyewear device 2002 and/or to other devices. These other devices may provide certain functions (e.g., tracking, localizing, depth mapping, processing, storage, etc.) to augmented-reality system 2000. In the embodiment of FIG. 20, neckband 2005 may include two acoustic transducers (e.g., 2020(I) and 2020(J)) that are part of the microphone array (or potentially form their own microphone subarray). Neckband 2005 may also include a controller 2025 and a power source 2035.

Acoustic transducers 2020(I) and 2020(J) of neckband 2005 may be configured to detect sound and convert the detected sound into an electronic format (analog or digital). In the embodiment of FIG. 20, acoustic transducers 2020(I) and 2020(J) may be positioned on neckband 2005, thereby increasing the distance between the neckband acoustic transducers 2020(I) and 2020(J) and other acoustic transducers 2020 positioned on eyewear device 2002. In some cases, increasing the distance between acoustic transducers 2020 of the microphone array may improve the accuracy of beamforming performed via the microphone array. For example, if a sound is detected by acoustic transducers 2020(C) and 2020(D) and the distance between acoustic transducers 2020(C) and 2020(D) is greater than, e.g., the distance between acoustic transducers 2020(D) and 2020(E), the determined source location of the detected sound may be more accurate than if the sound had been detected by acoustic transducers 2020(D) and 2020(E).

Controller 2025 of neckband 2005 may process information generated by the sensors on neckband 2005 and/or augmented-reality system 2000. For example, controller 2025 may process information from the microphone array that describes sounds detected by the microphone array. For each detected sound, controller 2025 may perform a direction-of-arrival (DOA) estimation to estimate a direction from which the detected sound arrived at the microphone array. As the microphone array detects sounds, controller 2025 may populate an audio data set with the information. In embodiments in which augmented-reality system 2000 includes an inertial measurement unit, controller 2025 may compute all inertial and spatial calculations from the IMU located on eyewear device 2002. A connector may convey information between augmented-reality system 2000 and neckband 2005 and between augmented-reality system 2000 and controller 2025. The information may be in the form of optical data, electrical data, wireless data, or any other transmittable data form. Moving the processing of information generated by augmented-reality system 2000 to neckband 2005 may reduce weight and heat in eyewear device 2002, making it more comfortable to the user.

Power source 2035 in neckband 2005 may provide power to eyewear device 2002 and/or to neckband 2005. Power source 2035 may include, without limitation, lithium-ion batteries, lithium-polymer batteries, primary lithium batteries, alkaline batteries, or any other form of power storage. In some cases, power source 2035 may be a wired power source. Including power source 2035 on neckband 2005 instead of on eyewear device 2002 may help better distribute the weight and heat generated by power source 2035.

As noted, some artificial-reality systems may, instead of blending an artificial reality with actual reality, substantially replace one or more of a user's sensory perceptions of the real world with a virtual experience. One example of this type of system is a head-worn display system, such as virtual-reality system 2100 in FIG. 21, that mostly or completely covers a user's field of view. Virtual-reality system 2100 may include a front rigid body 2102 and a band 2104 shaped to fit around a user's head. Virtual-reality system 2100 may also include output audio transducers 2106(A) and 2106(B). Furthermore, while not shown in FIG. 21, front rigid body 2102 may include one or more electronic elements, including one or more electronic displays, one or more inertial measurement units (IMUs), one or more tracking emitters or detectors, and/or any other suitable device or system for creating an artificial-reality experience.

Artificial-reality systems may include a variety of types of visual feedback mechanisms. For example, display devices in augmented-reality system 2000 and/or virtual-reality system 2100 may include one or more liquid crystal displays (LCDs), light emitting diode (LED) displays, microLED displays, organic LED (OLED) displays, digital light project (DLP) micro-displays, liquid crystal on silicon (LCoS) micro-displays, and/or any other suitable type of display screen. These artificial-reality systems may include a single display screen for both eyes or may provide a display screen for each eye, which may allow for additional flexibility for varifocal adjustments or for correcting a user's refractive error. Some of these artificial-reality systems may also include optical subsystems having one or more lenses (e.g., concave or convex lenses, Fresnel lenses, adjustable liquid lenses, etc.) through which a user may view a display screen. These optical subsystems may serve a variety of purposes, including to collimate (e.g., make an object appear at a greater distance than its physical distance), to magnify (e.g., make an object appear larger than its actual size), and/or to relay (to, e.g., the viewer's eyes) light. These optical subsystems may be used in a non-pupil-forming architecture (such as a single lens configuration that directly collimates light but results in so-called pincushion distortion) and/or a pupil-forming architecture (such as a multi-lens configuration that produces so-called barrel distortion to nullify pincushion distortion).

In addition to or instead of using display screens, some of the artificial-reality systems described herein may include one or more projection systems. For example, display devices in augmented-reality system 2000 and/or virtual-reality system 2100 may include microLED projectors that project light (using, e.g., a waveguide) into display devices, such as clear combiner lenses that allow ambient light to pass through. The display devices may refract the projected light toward a user's pupil and may enable a user to simultaneously view both artificial-reality content and the real world. The display devices may accomplish this using any of a variety of different optical components, including waveguide components (e.g., holographic, planar, diffractive, polarized, and/or reflective waveguide elements), light-manipulation surfaces and elements (such as diffractive, reflective, and refractive elements and gratings), coupling elements, etc. Artificial-reality systems may also be configured with any other suitable type or form of image projection system, such as retinal projectors used in virtual retina displays.

The artificial-reality systems described herein may also include various types of computer vision components and subsystems. For example, augmented-reality system 2000 and/or virtual-reality system 2100 may include one or more optical sensors, such as two-dimensional (2D) or 3D cameras, structured light transmitters and detectors, time-of-flight depth sensors, single-beam or sweeping laser rangefinders, 3D LiDAR sensors, and/or any other suitable type or form of optical sensor. An artificial-reality system may process data from one or more of these sensors to identify a location of a user, to map the real world, to provide a user with context about real-world surroundings, and/or to perform a variety of other functions.

The artificial-reality systems described herein may also include one or more input and/or output audio transducers. Output audio transducers may include voice coil speakers, ribbon speakers, electrostatic speakers, piezoelectric speakers, bone conduction transducers, cartilage conduction transducers, tragus-vibration transducers, and/or any other suitable type or form of audio transducer. Similarly, input audio transducers may include condenser microphones, dynamic microphones, ribbon microphones, and/or any other type or form of input transducer. In some embodiments, a single transducer may be used for both audio input and audio output.

In some embodiments, the artificial-reality systems described herein may also include tactile (i.e., haptic) feedback systems, which may be incorporated into headwear, gloves, body suits, handheld controllers, environmental devices (e.g., chairs, floormats, etc.), and/or any other type of device or system. Haptic feedback systems may provide various types of cutaneous feedback, including vibration, force, traction, texture, and/or temperature. Haptic feedback systems may also provide various types of kinesthetic feedback, such as motion and compliance. Haptic feedback may be implemented using motors, piezoelectric actuators, fluidic systems, and/or a variety of other types of feedback mechanisms. Haptic feedback systems may be implemented independent of other artificial-reality devices, within other artificial-reality devices, and/or in conjunction with other artificial-reality devices.

By providing haptic sensations, audible content, and/or visual content, artificial-reality systems may create an entire virtual experience or enhance a user's real-world experience in a variety of contexts and environments. For instance, artificial-reality systems may assist or extend a user's perception, memory, or cognition within a particular environment. Some systems may enhance a user's interactions with other people in the real world or may enable more immersive interactions with other people in a virtual world. Artificial-reality systems may also be used for educational purposes (e.g., for teaching or training in schools, hospitals, government organizations, military organizations, business enterprises, etc.), entertainment purposes (e.g., for playing video games, listening to music, watching video content, etc.), and/or for accessibility purposes (e.g., as hearing aids, visual aids, etc.). The embodiments disclosed herein may enable or enhance a user's artificial-reality experience in one or more of these contexts and environments and/or in other contexts and environments.

### EXAMPLE EMBODIMENTS

Example 1: A system may include a radar component and a controller configured to (1) cause the radar component to emit one or more radar signals and (2) cause the radar component to analyze one or more return signals (e.g., by identifying one or more features of an object toward which the radar signals were directed).

Example 2: The system of Example 1, where the radar component includes a transmitter, an antenna, and a receiver.

Example 3: The system of any of Examples land 2, where the radar component is configured to measure at least one of amplitude, phase, or frequency of the return signals.

Example 4: The system of any of Examples 1-3, where the radar component performs the analysis using a trained machine learning model.

Example 5: The system of any of Example 4, where the trained machine learning model is trained using radar return signals and camera data.

Example 6: The system of any of Examples 1-5, where the radar component is configured to identify one or more features in the object using feature extraction.

Example 7: The system of any of Example 6, where the object is or includes a user's face and/or body.

Example 8: The system of any of Examples 1-7, where the radar component is configured to sense a user's heartbeat by analyzing pulse or body movements.

Example 9: The system of any of Examples 1-8, where the system further includes one or more metallic reflectors configured to provide a broadened field of view for the radar component.

Example 10: A computer-implemented method including (1) causing a radar component to emit one or more radar signals and (2) causing the radar component to analyze one or more return signals (e.g., by identifying one or more features of an object toward which the radar signals were directed).

Example 11: The computer-implemented method of Example 10, where the radar component includes a transmitter, an antenna, and a receiver.

Example 12: The computer-implemented method of Examples 10 and 11, where the method further includes measuring at least one of amplitude, phase, or frequency of the return signals.

Example 13: The computer-implemented method of any of Examples 10-12, where analyzing the one or more return signals includes using a trained machine learning model to analyze the one or more return signals.

Example 14: The computer-implemented method of Example 13, where the trained machine learning model is trained using radar return signals and camera data.

Example 15: The computer-implemented method of any of Examples 10-14, where the radar component is configured to identify one or more features in the object using feature extraction.

Example 16: The computer-implemented method of Example 15, where the object is or includes a user's face and/or body.

Example 17: The computer-implemented method of any of Examples 10-16, where the method further includes sensing a user's heartbeat by analyzing pulse or body movements.

Example 18: An artificial realty device including a radar component configured to (1) emit one or more radar signals and (2) analyze one or more return signals (e.g., by identifying one or more features of an object toward which the radar signals were directed).

Example 19: The artificial reality device of Example 18, where the artificial reality device further includes metallic reflectors configured to focus the field of view of the radar component.

Example 20: The artificial reality device of Examples 18 and 19, where (1) the radar component is a radar sensor and (2) the return signals represent the radar signals returned to the radar component after having reflected off the object.

In particular embodiments, privacy settings may allow users to review and control, via opt in or opt out selections, as appropriate, how their user data may be collected, used, stored, shared, or deleted by the system or other entities (e.g., other users or third-party systems) fora particular purpose. The system may present users with an interface indicating what user data is being collected, used, stored, or shared by the system or other entities and for what purpose. Furthermore, the system may present users with an interface indicating how such user data is being collected, used, stored, or shared by particular processes of the system or other processes (e.g., internal research, advertising algorithms, machine-learning algorithms). Users may have to provide prior authorization before the system may collect, use, store, share, or delete any of their user data for any purpose.

Moreover, in particular embodiments, privacy policies may limit the types of user data that may be collected, used, or shared by particular processes of the system or other processes (e.g., internal research, advertising algorithms, machine-learning algorithms) for a particular purpose. The system may present users with an interface indicating the particular purpose for which user data is being collected, used, or shared. The privacy policies may ensure that only necessary and relevant user data is being collected, used, or shared for the particular purpose, and may prevent such user data from being collected, used, or shared for unauthorized purposes.

Still further, in particular embodiments, the collection, usage, storage, and sharing of any user data may be subject to data minimization policies, which may limit how such user data that may be collected, used, stored, or shared by the system, other entities (e.g., other users or third-party systems) or particular processes (e.g., internal research, advertising algorithms, machine-learning algorithms) for a particular purpose. The data minimization policies may ensure that only relevant and necessary user data may be accessed by such entities or processes for such purposes.

As detailed above, the computing devices and systems described and/or illustrated herein broadly represent any type or form of computing device or system capable of executing computer-readable instructions, such as those contained within the modules described herein. In their most basic configuration, these computing device(s) may each include at least one memory device (e.g., memory 224 and 226 in FIG. 2) and at least one physical processor (e.g., physical processors 228 and 230 in FIG. 2).

In some examples, the term “memory device” generally refers to any type or form of volatile or non-volatile storage device or medium capable of storing data and/or computer-readable instructions. In one example, a memory device may store, load, and/or maintain one or more of the modules described herein. Examples of memory devices include, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In some examples, the term “physical processor” generally refers to any type or form of hardware-implemented processing unit capable of interpreting and/or executing computer-readable instructions. In one example, a physical processor may access and/or modify one or more modules stored in the above-described memory device. Examples of physical processors include, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the modules described and/or illustrated herein may represent portions of a single module or application. In addition, in certain embodiments one or more of these modules may represent one or more software applications or programs that, when executed by a computing device, may cause the computing device to perform one or more tasks. For example, one or more of the modules described and/or illustrated herein may represent modules stored and configured to run on one or more of the computing devices or systems described and/or illustrated herein. One or more of these modules may also represent all or portions of one or more special-purpose computers configured to perform one or more tasks.

In addition, one or more of the modules described herein may transform data, physical devices, and/or representations of physical devices from one form to another. For example, one or more of the modules recited herein may receive data (e.g., radar data) to be transformed, transform the data, and output a result of the transformation, use the result of the transformation to perform a function, and store the result of the transformation. Additionally or alternatively, one or more of the modules recited herein may transform a processor, volatile memory, non-volatile memory, and/or any other portion of a physical computing device from one form to another by executing on the computing device, storing data on the computing device, and/or otherwise interacting with the computing device.

In some embodiments, the term “computer-readable medium” generally refers to any form of device, carrier, or medium capable of storing or carrying computer-readable instructions. Examples of computer-readable media include, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed. The various exemplary methods described and/or illustrated herein may also omit one or more of the steps described or illustrated herein or include additional steps in addition to those disclosed.

The preceding description has been provided to enable others skilled in the art to best utilize various aspects of the exemplary embodiments disclosed herein. This exemplary description is not intended to be exhaustive or to be limited to any precise form disclosed. Many modifications and variations are possible without departing from the spirit and scope of the present disclosure. The embodiments disclosed herein should be considered in all respects illustrative and not restrictive. Reference should be made to the appended claims and their equivalents in determining the scope of the present disclosure.

Unless otherwise noted, the terms “connected to” and “coupled to” (and their derivatives), as used in the specification and claims, are to be construed as permitting both direct and indirect (i.e., via other elements or components) connection. In addition, the terms “a” or “an,” as used in the specification and claims, are to be construed as meaning “at least one of.” Finally, for ease of use, the terms “including” and “having” (and their derivatives), as used in the specification and claims, are interchangeable with and have the same meaning as the word “comprising.”

What is claimed is:

1. A system comprising:
at least one radar component; and
at least one controller that is configured to:
cause the radar component to emit one or more radar signals; and
cause the radar component to analyze one or more return signals, wherein the analysis includes identifying one or more features of an object toward which the radar signals were directed.

2. The system of claim 1, wherein the radar component includes a transmitter, an antenna, and a receiver.

3. The system of claim 1, wherein the radar component is configured to measure at least one of amplitude, phase, or frequency of the return signals.

4. The system of claim 1, wherein the radar component performs the analysis using a trained machine learning model.

5. The system of claim 4, wherein the trained machine learning model is trained using radar return signals and camera data.

6. The system of claim 1, wherein the radar component is configured to identify one or more features in the object using feature extraction.

7. The system of claim 6, wherein the object comprises a user's face or body.

8. The system of claim 1, wherein the radar component is configured to sense a user's heartbeat by analyzing pulse or body movements.

9. The system of claim 1, further comprising one or more metallic reflectors configured to provide a broadened field of view for the radar component.

10. A computer-implemented method comprising:
causing a radar component to emit one or more radar signals; and
causing the radar component to analyze one or more return signals, wherein the analysis includes identifying one or more features of an object toward which the radar signals were directed.

11. The computer-implemented method of claim 10, wherein the radar component includes a transmitter, an antenna, and a receiver.

12. The computer-implemented method of claim 10, further comprising measuring at least one of amplitude, phase, or frequency of the return signals.

13. The computer-implemented method of claim 10, wherein analyzing the one or more return signals comprising using a trained machine learning model to analyze the one or more return signals.

14. The computer-implemented method of claim 13, wherein the trained machine learning model is trained using radar return signals and camera data.

15. The computer-implemented method of claim 10, wherein the radar component is configured to identify one or more features in the object using feature extraction.

16. The computer-implemented method of claim 15, wherein the object comprises a user's face or body.

17. The computer-implemented method of claim 10, further comprising sensing a user's heartbeat by analyzing pulse or body movements.

18. A method comprising:
heating an embossing element;
moving the embossing element proximate to a crystalline material; and
transferring a source of patterned energy from the embossing element to the crystalline material.

19. The method of claim 18, wherein the embossing element comprises a laser.

20. The method of claim 18, wherein the embossing element comprises a photon beam.
